(19) United States
(12) Patent Application Publication
(10) Pub. No.: US 2013/0079394 A1
(43) Pub. Date: Mar. 28, 2013

(54) Indirect modeling of new repellent molecules active against insects, acarids, and other arthropods

(71) Applicants: Jean Delaveau, Saint Nicolas de Macherin (FR); Pierre Nicolas Stebe, Forbach (FR); Nathalie Marchand-Geneste, Talence Cedex (FR)

(72) Inventors: Jean Delaveau, Saint Nicolas de Macherin (FR); Pierre Nicolas Stebe, Forbach (FR); Nathalie Marchand-Geneste, Talence Cedex (FR)

(73) Assignees: CNRS (CENTRE NATIONAL RECHERCHE SCIENTIFIQUE), Paris cedex (FR); MERIAL LIMITED, Duluth, GA (US)

(21) Appl. No.: 13/624,641

(22) Filed: Sep. 21, 2012

Related U.S. Application Data
(60) Provisional application No. 61/538,425, filed on Sep. 23, 2011.

(51) Int. Cl. C07D 311/14; A01P 17/00; C07D 311/18; C07D 311/16; A01N 43/16; C07D 311/46

(52) U.S. Cl. 514/457; 549/289; 549/285; 549/283; 549/287

(57) ABSTRACT

This invention relates to novel coumarin derivatives, formulations comprising same, and to methods of making and using these compounds and formulations, which are useful as repellents against insects and/or pests. The compounds also prevent illness and disease caused by insect/pest-borne vectors, and provide safer, more effective alternatives to existing repellents. This invention also relates to novel methods for modeling and/or predicting the repellency of unknown compounds.

### INCORPORATION BY REFERENCE

This application claims priority to U.S. provisional patent application Ser. No. 61/538,425, filed on Sep. 23, 2011, the disclosure of which is incorporated herein by reference.

### FIELD OF THE INVENTION

The present invention relates to novel coumarin derivatives, a method for making such compounds, and a method of repelling insects and pests away from animals, including humans. The present invention has particular, though not sole, application to repelling insects including flies and mosquitoes.

### BACKGROUND

Diseases such as malaria, dengue fever, chikungunya, yellow fever, Rift valley fever, Nil fever, etc (MS Fradin, 1998), are responsible for significant morbidity and mortality in humans and livestock in many parts of the world (T Katz et al., 2008). Bloodsucking arthropods are considered to be the principal vector of transmission for these diseases. Among them, mosquitoes and ticks are responsible for most of these pathologies. In order to prevent the transmission of the pathogens from the infected hosts, different solutions are employed: either an insecticide device or substance is used bed nets, insect traps or spraying of insecticides (DOT was the most common product in the 60-70s), or a repellent substance (citronella, Picaridine, etc.) can be applied on the clothes or on the skin.

Repellents are used to keep pests, including insects and acarids, away from animals including humans. In addition to being a nuisance, pests are vectors for many disease/disorder-causing agents including parasites, bacteria, and virus. Several known repellents are presented in Table 1. An ideal repellent should have prolonged efficacy, act against multiple different species, be non-toxic, be stable and inert to common plastics and clothing, be economically viable, and should be odorless or have a non-offensive odor (T Katz et al., 2008).

TABLE 1
Commonly known repellents

| | | Mole- | |
|---|---|---|---|
| | | cular | |
| Name | Molecule | mass | Formula |
| Diethyl- toluamide (DEET) | 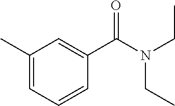 | 191.13 | C₁₂H₁₇NO |
| Picaridine | 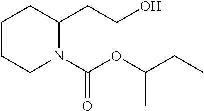 [SMILES: CCC(C)OC(=O)N1CCCCC1CCO] | 229.17 | C₁₂H₂₃NO₃ |
| Benzyl benzoate | 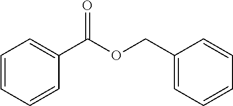 [SMILES: O=C(OCc1ccccc1)c1ccccc1] | 212.08 | C₁₄H₁₂O₂ |
| Coumarin | 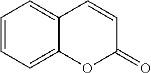 [SMILES: O=c1ccc2ccccc2o1] | 146.15 | C₉H₆O₂ |

N,N-Diethyl-meta-toluamide, abbreviated DEET, is a slightly yellow oil. It is the most common active ingredient in insect repellents. It is intended to be applied to the skin or to clothing, and is primarily used to repel mosquitoes. In particular, DEET is known to provide some protection against tick bites and mosquito bites, which can both transmit disease. DEET was historically believed to work by blocking insect olfactory receptors for 1-octen-3-ol, a volatile substance that is contained in human sweat and breath. The prevailing theory was that DEET effectively “blinded” the insect's senses so that the biting/feeding instinct is not triggered by humans or other animals which produce these chemicals. DEET does not appear to affect the insect's ability to smell carbon dioxide, as had been suspected earlier (Petherick et al., 2008; Ditzen et al., 2008) However, more recent evidence shows that DEET serves as a true repellent in that mosquitoes intensely dislike the smell of the chemical repellent (Syed and Leal, 2008). A type of olfactory receptor neuron in special antennal sensilla of mosquitoes that is activated by DEET as well as other known insect repellents such as eucalyptol, linalool, and thujone has been identified. Moreover, in a behavioral test DEFT had a strong repellent activity in the absence of body odor attractants such as 1-octen-3-ol, lactic acid, or carbon dioxide.

DEET Health Effects.

As a precaution, manufacturers advise that DEET products should not be used under clothing or on damaged skin, and that preparations be washed off after they are no longer needed or between applications. As a precaution, manufacturers advise that DEET products should not be used under clothing or on damaged skin, and that preparations be washed off after they are no longer needed or between applications. DEET can act as an irritant; in rare cases, it may cause skin reactions (CDC Report). In the DEET Reregistration Eligibility Decision (RED), the US Environmental Protection Agency (EPA) reported 14 to 46 cases of potential DEET-associated seizures, including 4 deaths. The EPA states: “ . . . it does appear that some cases are likely related to DEET toxicity,” but observed that with 30% of the US population using DEET, the likely seizure rate is only about one per 100 million users (US EPA, 1998).

In order to develop improved repellents, some research has been performed as regards quantitative structure activity relationship (QSAR) models to predict a molecule's repellency potential (MVS Suryanarayana, 1991). A regression equation was obtained (based on data using Aedes aegypti), which described the protection time (PT) as being a function of the vapor pressure (VP), the molecular length (ML) and the logarithm of the octanol water partition coefficient (log P). The equation is as follows, with constants A, B, C and D:

PT=A×log P+B×log Vₚ+C×D (Eq.1)

Equation 1 indicates that the repellent properties of a compound are dependant of its lipophilicity, its volatility and its three-dimensional shape. Importantly, when this model was applied to all 40 study molecules, its strength was quite low (R²=0.55). However, the coefficient was increased to 0.98 when the model was applied to a smaller subset of molecules (n=5). The results generally suggested a good repellent compound should adhere to the following criteria: 1.5<log P<2.5; ML>10.6 Å; and Vp≈0.2 mmHg. Other groups built on the work of Suryanarayana, including Ma (D Ma et al., 1999) and Wesley (????). Ma teaches relationships concerning the electrostatic properties of repellents, and Wesley discusses the link between repellency and molecule size. Further, a model generated with the CODESSA PRO software (Katritzky, 2002) gave the best result as of the filing of the instant disclosure (R²=0.78, F=23.9, s²=0.51, t test=−2.91, R²CV=0.70). The model's equation is given in Eq. 2 where J is the kinetic moment; SIC the structural information content, χ the Kier and Hall index and μ the dipole moment.

PT=−86.2×J−0.93×SIC−0.99×χ−2.66×μ+21.1 (Eq.2)

Thus, parameters depicting steric and electronic properties may play an important role to play in the prediction of the repellency. The results obtained by Suryanarayana and Katritzky differ primarily because 1) the experimentally measured protection time was not the same, and 2) different molecular descriptors were calculated in the more recent study. Pharmacophore models have also been generated to predict repellency potential (please see U.S. Pat. No. 7,897,162, to Gupta; Bhattacharjee et al., 2005; and ACCELRYS, Catalys Software Inc.).

Up until now, all existing models for repellency have been based on protection time value, which is different for each. Moreover, the environmental parameters in which the animals are studied are frequently different. As a consequence, data produced using different models cannot be combined/compared. Recently, a molecule dataset has been produced, wherein the repellency of novel coumarin derivatives was evaluated in vivo (USSN 61,501,485, to Merial Limited, and herein incorporated by reference in its entirety). Coumarin (2H-chromen-2-one) is a pleasantly fragrant benzopyrone phenylpropanoid chemical compound found in many plants, notably in high concentration in the tonka bean (Dipteryx odorata), vanilla grass (Anthoxanthum odoratum), sweet woodruff (Galium odoratum), mullein (Verbascum spp.), sweet grass (Hierochloe odorata), Cassia cinnamon (Cinnamomum aromaticum) and sweet clover. The numerical value obtained was not a protection time but rather an index ranging from −1 to 1 (with 1 being highly repellent). This number was calculated for each experiment and is function of a number of flies. It remains to prioritize further investigation of compounds having the greatest chance of being effective repellents.

There are clear, long-felt, and unmet needs for new repellents having improved safety and efficacy profiles. It is therefore an object of the instant invention to provide a model for reliably predicting the potential repellency of candidate compounds. It is a further object to provide novel derivatives of coumarin having improved safety and efficacy as compared with currently available repellents.

It is expressly noted that citation or identification of any document in this application is not an admission that such document is available as prior art to the present invention. Any foregoing applications, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### SUMMARY OF THE INVENTION

A first aspect of the invention is to provide novel QSAR models for predicting the repellency potential of compounds based, in part, on their molecular structure. The models may take the form of a pharmacophore, a linear equation or a classification tree. Each model may express the repellency potential as a function of chemical features and descriptors. A “molecular descriptor” is defined herein as “the final result of logic and mathematical procedure which transforms chemical information encoded within a symbolic representation of a molecule into a useful number or the result of some standardized experiment.”

In an embodiment, the model may be used to predict the repellency potential of compounds for which no repellency data has been generated previously.

In an embodiment, the molecular descriptors may include, but not be limited to the following: steric, physicochemical properties, topological, lipophilic, and electronic.

In another embodiment, descriptors including the substituent in position 4, 6, or 7 on coumarin, or the lateral chain length of the substituent may be used in modeling repellency of coumarin derivatives.

In yet another embodiment, the compounds are known repellents, including, but not limited to DEET, picaridine, and benzyl benzoate.

In an embodiment, the compounds are novel coumarin derivatives.

A second aspect of the invention is to provide a method for modeling the repellency potential of a compound. In some embodiments, the compound's repellency potential. In some embodiments, the repellency has not been previously evaluated.

A third aspect of the invention is to provide novel compounds that are based upon the bicyclic compound coumarin, and are active in repelling pests, including insects and acarids, that are a burden to animals including humans. In some embodiments, the compounds are effective repellents against mosquitoes, flies, ticks, and fleas.

The invention is also directed toward a method of protecting an animal (e.g. a mammal or bird) against pests by administering a effective repelling amount of the compositions of the invention. Animals which can be treated include but are not limited to chickens/avians, humans, cats, dogs, cattle, cows, deer, goats, horses, llamas, pigs, sheep and yaks. In one embodiment of the invention, the animals treated are canines, felines, or humans.

In one embodiment, the present invention provides coumarin derivative compounds of formula (I) or (Ia) shown below:

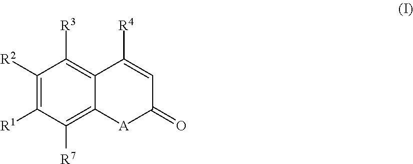

or a veterinarily or pharmaceutically acceptable salt thereof; wherein R¹, R², R³, and R⁴ independently include H, C, OR⁵, CR⁵, OCC(═O)N(R⁵)(R⁸), CC(═O)N(R⁵)(R⁸), and wherein A is O, or the ring is opened at A, thus forming compounds according to formula (Ia):

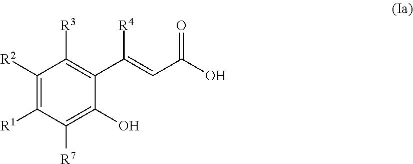

and wherein additional meanings of variables R¹, R², R³, R⁴, R⁵, R⁶ and R⁸ are as described below. The invention also provides veterinary and pharmaceutical compositions comprising the inventive compounds, or salts thereof, in combination with a veterinarily or pharmaceutically acceptable carrier or diluent.

The inventive compounds and compositions comprising the compounds are highly effective for the repulsion of pests. Accordingly, the present invention provides methods for repelling pests away from animals, including humans, comprising applying a repulsive effective amount of a compound of formula (I) or (Ia), or a veterinarily or pharmaceutically acceptable salt thereof, to the animal or human, or its surroundings.

It is an object of the invention to not encompass within the invention any previously known product, process of making the product, or method of using the product such that the Applicants reserve the right to this invention and hereby disclose a disclaimer of any previously known product, process, or method.

It is noted that in this disclosure and particularly in the claims, terms such as “comprises”, “comprised”, “comprising” and the like can have the meaning attributed to such terms in U.S. Patent law; e.g., they can mean “includes”, “included”, “including”, and the like; and that terms such as “consisting essentially of” and “consists essentially of” have the meaning ascribed to them by U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF DRAWINGS

A full and enabling disclosure of the present invention, including the best mode thereof, to one of ordinary skill in the art, is set forth more particularly in the remainder of the specification, including reference to the accompanying figures, wherein:

FIG. 1A is a representation of the Pharmacophore model obtained with the reduced number of coumarin derivatives set. Hydrogen bond acceptors are in green and hydrophobic features are shown in blue;

FIG. 1B is the model of FIG. 1A presented with a non-substituted coumarin

FIG. 2 is a graph of experimental repellency vs. estimate repellency for the pharmacophore model;

FIG. 3 is a representation of the experimental repellency index (RI) vs. the predicted repellency index. Four descriptors' model generated on the 57 coumarins set;

FIG. 4 is a projection of the repellency index from the 50 coumarins with a positive repellency index;

FIG. 5 depicts the selection classification tree for the complete set of coumarins (Xi are coefficients);

FIG. 6 is a plot of the results of the linear model generated on the reduced set of 34 coumarins (RI>0.5) n_{training}=23, nₜₑₛₜ=11;

FIG. 7 presents results for the 3 descriptor linear model generated on the reduced set of 28 coumarins (RI>0.7) n_{training}=19, nₜₑₛₜ=9;

FIG. 8 presents results for the 4 descriptor linear model generated on the reduced set of 28 coumarins (RI>0.7) n_{training}=19, nₜₑₛₜ=9;

FIG. 9 is a first classification tree obtained with the reduced set of coumarins with a repellency index higher than 0.5 (n_{training}=23, nₜₑₛₜ=11);

FIG. 10 is a second classification tree obtained with the reduced set of coumarins with a repellency index higher than 0.5 (n training=23, n test=11);

FIG. 11 is a classification tree obtained with the reduced set of coumarins with a repellency index higher than 0.7 (training=19, n test 9);

FIG. 12 is a diagram representing the way the activities of the new molecules were predicted.

### DETAILED DESCRIPTION OF THE INVENTION

The present invention provides novel methods and models useful for predicting the repellency potential of compounds. In some embodiments, the invention provides novel QSAR models for predicting the repellency potential of compounds based, in part, on their molecular structure. The models may take the form of a pharmacophore, a linear equation or a classification tree. Each model may express the repellency potential as a function of chemical features and descriptors, as defined above.

The present invention also provides for coumarin derivative compounds with insect and pest repellent activity, or pharmaceutically/veterinarily acceptable or pharmaceutically acceptable salts thereof, and compositions comprising the compounds or salts for the repulsion of insects or other pests away from an animal or a human. An important aspect of the invention is to provide coumarin derivative compounds with high repellent activity against pests, particularly though not solely insects, and improved safety to the user, the environment, and the animal.

The invention includes at least the following features:

(a) In an embodiment, the invention provides for novel models useful for predicting the repellency of small molecule compounds.

(b) In another embodiment, the invention provides novel compounds of formula (I) or (Ia), or veterinarily or pharmaceutically acceptable salts thereof, which is a repellent of animal pests, including insects and acarids;

(c) veterinary and pharmaceutical compositions for repelling pests comprising repellent effective amount of the compounds of formula (I) or (Ia), or veterinarily or pharmaceutically acceptable salts thereof, in combination with a veterinarily or pharmaceutically acceptable carrier or diluent;

(d) veterinary and pharmaceutical compositions for repelling pests comprising a repellent effective amount of the compounds of the invention, or veterinarily or pharmaceutically acceptable salts thereof, in combination with one more other active agent, including other repellents, antiparasitics, and a veterinarily or pharmaceutically acceptable carrier or diluent;

(e) methods for repelling pests, including insects and acarids, away from an animal, including a human, are provided, which methods comprise administering a repellent effective amount of a compound of formula (I) or (Ia), or veterinarily acceptable salts thereof, to the animal in need thereof;

(f) methods for the prevention of infestation/infection and/or the reduction of transmission of a pest-borne pathogen to animals, including humans, which comprise administering a repellent effective amount of a compound of formula (I) or (Ia), or veterinarily or pharmaceutically acceptable salts thereof, to the animal in need thereof, thereby preventing infection/infestation and/or reducing the transmission of pest-borne pathogens to animals, including humans;

(g) methods for controlling pests at a locus (e.g. by repelling pests away from a locus), comprising administering or applying a repellent effective amount of a compound of formula (I), or veterinarily or pharmaceutically acceptable salts thereof, to the locus;

(h) use of the compounds of formula (I) or (Ia), or veterinarily acceptable salts thereof, in the manufacture of a veterinary or pharmaceutical medicament for repelling pests, including insects and acarids; and

(i) processes for the preparation of the compounds of formula (I) or (Ia).

### DEFINITIONS

Terms used herein will have their customary meanings in the art unless specified. The organic moieties mentioned in the definitions of the variables of formula (I) are like the term halogen—i.e., collective terms for individual listings of the individual group members. The prefix Cₙ-Cₘ indicates in each case the possible number of carbon atoms in the group.

The term “alkyl” refers to saturated straight, branched, cyclic, primary, secondary or tertiary hydrocarbons, including those having 1 to 12 atoms. In some embodiments, alkyl groups will include C₁-C₁₀, C₁-C₈, C₁-C₆ or C₁-C₄ alkyl groups. Examples of C₁-C₁₀ alkyl include, but are not limited to, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl and their isomers. C₁-C₄-alkyl means for example methyl, ethyl, propyl, 1-methylethyl, butyl, 1-methylpropyl, 2-methylpropyl or 1,1-dimethylethyl.

Cyclic alkyl groups, which are encompassed by alkyls, may be referred to as “cycloalkyl” and include those with 3 to 10 carbon atoms having single or multiple fused rings. Non-limiting examples of cycloalkyl groups include adamantyl, cyclopropyl, cyclobutyl, cyclopentyl, cyclohexyl, cycloheptyl, cyclooctyl and the like.

The alkyl and cycloalkyl groups described herein can be unsubstituted or substituted with one or more moieties selected from the group consisting of alkyl, halo, haloalkyl, hydroxyl, carboxyl, acyl, acyloxy, amino, alkyl- or dialkylamino, amido, arylamino, alkoxy, aryloxy, nitro, cyano, azido, thiol, imino, sulfonic acid, sulfate, sulfonyl, sulfonyl, sulfinyl, sulfamonyl, ester, phosphoryl, phosphinyl, phosphoryl, phosphine, thioester, thioether, acid halide, anhydride, oxime, hydrozine, carbamate, phosphonic acid, phosphate, phosphonate, or any other viable functional group that does, not inhibit the biological activity of the compounds of the invention, either unprotected, or protected as necessary, as known to those skilled in the art, for example, as taught in Greene, et al., Protective Groups in Organic Synthesis, John Wiley and Sons, Third Edition, 1999, hereby incorporated by reference.

The term “alkenyl” refers to both straight and branched carbon chains which have at least one carbon-carbon double bond. In some embodiments, alkenyl groups may include C₂-C₁₂ alkenyl groups. In other embodiments, alkenyl includes C₂-C₁₀, C₂-C₈, C₂-C₆ or C₂-C₄ alkenyl groups. In one embodiment of alkenyl, the number of double bonds is 1-3; in another embodiment of alkenyl, the number of double bonds is one. Other ranges of carbon-carbon double bonds and carbon numbers are also contemplated depending on the location of the alkenyl moiety on the molecule. “C₂-C₁₀-alkenyl” groups may include more than one double bond in the chain. Examples include, but are not limited to, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

“Cycloalkenyl” refers to monovalent cyclic alkenyl groups of from 4 to 10 carbon atoms, preferably 5 to 8 carbon atoms, having single or multiple fused rings which fused rings may or may not be cycloalkenyl provided that the point of attachment is to a cycloalkenyl ring atom. Examples of cycloalkenyl groups include, by way of example, cyclopenten-4-yl, cyclooctene-5-yl and the like. Alkenyl and cycloalkenyl groups may be unsubstituted or substituted with one or more substituents as described for alkyl above.

“Alkynyl” refers to both straight and branched carbon chains which have at least one carbon-carbon triple bond. In one embodiment of alkynyl, the number of triple bonds is 1-3; in another embodiment of alkynyl, the number of triple bonds is one. In some embodiments, alkynyl groups include from 2 to 12 carbon atoms. In other embodiments, alkynyl groups may include C₂-C₁₀, C₂-C₈, C₂-C₆ or C₂-C₄ alkynyl groups. Other ranges of carbon-carbon triple bonds and carbon numbers are also contemplated depending on the location of the alkenyl moiety on the molecule. For example, the term “C₂-C₁₀-alkynyl” as used herein refers to a straight-chain or branched unsaturated hydrocarbon group having 2 to 10 carbon atoms and containing at least one triple bond, such as ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl or 4-methylpent-2-yn-5-yl and the like.

The term “haloalkyl” refers to an alkyl group, as defined herein, which is substituted by one or more halogen atoms. For example C₁-C₄-haloalkyl includes, but is not limited to, chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like.

The term “fluoroalkyl” as used herein refers to an alkyl in which one or more of the hydrogen atoms is replaced with fluorine atoms, for example difluoromethyl, trifluoromethyl, 1-fluoroethyl, 2-fluoroethyl, 2,2-difluoroethyl, 2,2,2-trifluoroethyl, 1,1,2,2-tetrafluoroethyl or pentafluoroethyl.

The term “haloalkenyl” refers to an alkenyl group, as defined herein, which is substituted by one or more halogen atoms.

The term “haloalkynyl” refers to an alkynyl group, as defined herein, which is substituted by one or more halogen atoms.

“Alkoxy” refers to alkyl-O—, wherein alkyl is as defined above. Similarly, the terms “alkenyloxy,” “alkynyloxy,” “haloalkoxy,” “haloalkenyloxy,” “haloalkynyloxy;” “cycloalkoxy,” “cycloalkenyloxy,” “halocycloalkoxy,” and “halocycloalkenyloxy” refer to the groups alkenyl-O—, alkynyl-O—, haloalkyl-O—, haloalkenyl-O—, haloalkynyl-O—, cycloalkyl-O—, cycloalkenyl-O—, halocycloalkyl-O— and halocycloalkenyl-O—, respectively, wherein alkenyl, alkynyl, haloalkyl, haloalkenyl, haloalkynyl, cycloalkyl, cycloalkenyl, halocycloalkyl, and halocycloalkenyl are as defined above. Examples of C₁-C₆-alkoxy include, but are not limited to, methoxy, ethoxy, OCH₂—C₂H₅, OCH(CH₃)₂, n-butoxy, OCH(CH₃)—C₂H₅, OCH₂—CH(CH₃)₂, OC(CH₃)₃, n-pentoxy, 1-methylbutoxy, 2-methylbutoxy, 3-methylbutoxy, 1,1-dimethylpropoxy, 1,2-dimethylpropoxy, 2,2-dimethyl-propoxy, 1-ethylpropoxy, n-hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, 1-ethyl-2-methylpropoxy and the like.

“Aryl” refers to a monovalent aromatic carbocyclic group of from 6 to 14 carbon atoms having a single ring or multiple fused rings. Aryl groups include, but are not limited to, phenyl, biphenyl, and naphthyl. In some embodiments aryl includes tetrahydronapthyl, phenylcyclopropyl and indanyl. Aryl groups may be unsubstituted or substituted by one or more moieties selected from halogen, cyano, nitro, hydroxy, mercapto, amino, alkyl, alkenyl, alkynyl, cycloalkyl, cycloalkenyl, haloalkyl, haloalkenyl, haloalkynyl, halocycloalkyl, halocycloalkenyl, alkoxy, alkenyloxy, alkynyloxy, haloalkoxy, haloalkenyloxy, haloalkynyloxy, cycloalkoxy, cycloalkenyloxy, halocycloalkoxy, halocycloalkenyloxy, alkylthio, haloalkylthio, cycloalkylthio, halocycloalkylthio, alkylsulfinyl, alkenylsulfinyl, alkynyl-sulfinyl, haloalkylsulfinyl, haloalkenylsulfinyl, haloalkynylsulfinyl, alkylsulfonyl, alkenylsulfonyl, alkynylsulfonyl, haloalkyl-sulfonyl, haloalkenylsulfonyl, haloalkynylsulfonyl, alkylamino, alkenylamino, alkynylamino, di(alkyl)amino, di(alkenyl)-amino, di(alkynyl)amino, or trialkylsilyl.

The term “aralkyl” refers to an aryl group that is bonded to the parent compound through a diradical alkylene bridge, (—CH₂—)ₙ, where n is 1-12 and where “aryl” is as defined above.

“Heteroaryl” refers to a monovalent aromatic group of from 1 to 15 carbon atoms, preferably from 1 to 10 carbon atoms, having one or more oxygen, nitrogen, and sulfur heteroatoms within the ring, preferably 1 to 4 heteroatoms, or 1 to 3 heteroatoms. The nitrogen and sulfur heteroatoms may optionally be oxidized. Such heteroaryl groups can have a single ring (e.g., pyridyl or furyl) or multiple fused rings provided that the point of attachment is through a heteroaryl ring atom. Examples of heteroaryls include pyridyl, piridazinyl, pyrimidinyl, pyrazinyl, triazinyl, pyrrolyl, indolyl, quinolinyl, isoquinolinyl, quinazolinyl, quinoxalinnyl, furanyl, thiophenyl, furyl, pyrrolyl, imidazolyl, oxazolyl, isoxazolyl, isothiazolyl, pyrazolyl benzofuranyl, and benzothiophenyl. Heteroaryl rings may be unsubstituted or substituted by one or more moieties as described for aryl above.

“Heterocyclyl,” “heterocyclic” or “heterocyclo” refers to fully saturated or unsaturated, cyclic groups, for example, 3 to 7 membered monocyclic, 7 to 11 membered bicyclic, or 10 to 15 membered tricyclic ring systems, which have one or more oxygen, sulfur or nitrogen heteroatoms in ring, preferably 1 to 4 or 1 to 3 heteroatoms. The nitrogen and sulfur heteroatoms may optionally be oxidized and the nitrogen heteroatoms may optionally be quaternized. The heterocyclic group may be attached at any heteroatom or carbon atom of the ring or ring system and may be unsubstituted or substituted by one or more moieties as described for aryl groups above.

Exemplary monocyclic heterocyclic groups include, but are not limited to, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridinyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydno-1,1-dioxothienyl, triazolyl, triazinyl, and the like.

Exemplary bicyclic heterocyclic groups include, but are not limited to, indolyl, benzothiazolyl, benzoxazolyl, benzodioxolyl, benzothienyl, quinuclidinyl, quinolinyl, tetra-hydroisoquinolinyl, isoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), tetrahydroquinolinyl and the like.

The term “alkylthio” or “alkylsulfanyl” refers to alkyl-S—, where “alkyl” is as defined above. In some embodiments, the alkyl component of the alkylthio group will include C₁-C₁₀, C₁-C₈, C₁-C₆ or C₁-C₄ alkyl groups. For example, C₁-C₄-alkylthio include, but are not limited to, methylthio, ethylthio, propylthio, 1-methylethylthio, butylthio, 1-methylpropylthio, 2-methylpropylthio or 1,1-dimethylethylthio.

Similarly, the terms “haloalkylthio,” “cycloalkylthio,” “halocycloalkylthio” refer to the groups —S-haloalkyl, —S-cycloalkyl, and —S-halocycloalkyl, respectively, where the terms “haloalkyl,” “cycloalkyl,” and “halocycloalkyl” are as defined above.

The term “alkylsulfinyl” refers to the group alkyl-S(═O)—, where “alkyl” is as defined above. In some embodiments, the alkyl component in alkylsulfinyl groups will include C₁-C₁₂, C₁-C₁₀, C₁-C₈, C₁-C₆ or C₁-C₄ alkyl groups. Examples include, but are not limited to, —SO—CH₃, —SO—C₂H₅, n-propylsulfinyl, 1-methylethylsulfinyl, n-butylsulfinyl, 1-methylpropylsulfinyl, 2-methylpropylsulfinyl, 1,1-dimethylethylsulfinyl, n-pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, n-hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl.

Similarly, the terms “alkenylfulfinyl,” “alkynylsulfinyl,” “haloalkylsulfinyl,” “haloalkenylsulfinyl,” and “haloalkynylsulfinyl” refer to the groups alkenyl-S(═O)—, alkynyl-S(═O)—, and haloalkyl-S(═O)—, haloalkenyl-S(═O)—, and haloalkynyl-S(═O)—, where the terms “alkenyl,” “alkynyl,” “haloalkyl,” “haloalkenyl,” and “haloalkynyl” are as defined above.

The term “alkylsulfonyl” refers to the group alkyl-S(═O)₂—, where the term “alkyl” is as defined above. In, some embodiments, the alkyl component in alkylsulfonyl groups will include C₁-C₁₂, C₁-C₁₀, C₁-C₈, C₁-C₆ or C₁-C₄ alkyl groups. Examples include, but are not limited to, —SO₂—CH₃, —SO₂—C₂H₅, n-propylsulfonyl, —SO₂—CH(CH₃)₂, n-butylsulfonyl, 1-methylpropylsulfonyl, 2-methylpropylsulfonyl, —SO₂—C(CH₃)₃, n-pentylsulfonyl, 1-methyl butylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, n-hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl and the like.

The terms “alkenylfulfonyl,” “alkynylsulfonyl,” “haloalkylsulfonyl,” “haloafkenylsulfonyl,” and “haloalkynylsulfonyl” refer to the groups alkenyl-S(═O)₂—, alkynyl-S(═O)₂—, and haloalkyl-S(═O)₂—, haloalkenyl-S(═O)₂—, and haloalkynyl-S(═O)₂—, where the terms “alkenyl,” “alkenyl,” “haloalkyl,” “haloalkenyl,” and “haloalkynyl” are as defined above.

The terms “alkylamino,” “dialkylamino,” “alkenylamino,” “alkynylamino,” “di(alkenyl)amino,” and “di(alkynyl)amino” refer to the groups —NH(alkyl), —N(alkyl)₂, —NH-(alkenyl), —N(alkenyl)₂ and —N(alkynyl)₂, where the terms “alkyl,” “alkenyl,” and “alkynyl” are as defined above. In some embodiments, the alkyl component in alkylamino or dialkylamino groups will include C₁-C₁₂, C₁-C₁₀, C₁-C₈, C₁-C₆ or C₁-C₄ alkyl groups.

The term “trialkylsilyl” refers to the group —Si(alkyl)₃, where the group is bonded to the parent compound at the silicon atom.

### Compounds of the Invention:

In certain embodiments, the compounds of the invention are useful in veterinary applications, including for repelling pests, including insects and acarids, away from an animal. In other embodiments, the inventive compounds are useful in pharmaceutical, or veterinary applications for repelling insects or acarids.

In one embodiment the invention provides a coumarin derivative of formula (I), or a veterinarily or pharmaceutically acceptable salt thereof:

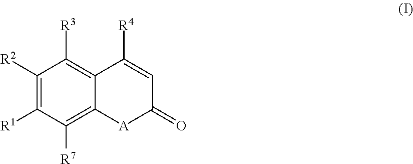

wherein

R¹, R², R³, and R⁴ independently include H, C, OR^{S}, CR⁵, OCC(═O)N(R⁵)(R⁸), CC(═O)N(R⁵)(R⁸), alkyl, aryl, aralkyl, heteroaryl, alcohols, amine, aldehyde, heterocyclyl, or salts of amines and carboxylates; wherein A is O, or the ring is opened at A, thus forming compounds according to formula (Ia):

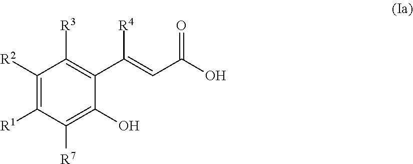

In an embodiment of the invention the compound of formula (I) or (Ia) is selected from the compounds described in Tables 2 and 9.

In another embodiment, the compound of formula (I) or (Ia) is PN10001, PN10002, PN10003, PN10004, PN10005, PN10006, PN10007, PN10008, PN10009, PN10010, NG1, NG2, NG3, NG4, NG5, NG6, NG7, NG8, PN10011, PN10012, PN10013, PN10014, PN10015, PN10016, PN10017, PN10018, PN10019, PN10020, PN10021, PN10022, PN10031, PN10032, PN10033, PN1.0034, PN10035, PN10036, PN10037, PN10038, PN10039, PN10040, PN10041, PN10042, PN10043, PN10044, PN10045, PN10046, PN10047, PN10048, PN10049, PN10050, PN10051, PN10052, PN10053, PN10054, PN10055, or PN10056.

In another embodiment, the compound of formula (I) or (Ia) is EV04016, EV04024, EV04030, EV04032, EV04036, EV04054, EV04058, EV04062, EV04070, EV04084, EV04090, EV04094, EV04114, EV04122, EV04188, EV05056, EV05084, EV05088, EV05096, EV05118, EV05120, EV05134, EV05138, EV05144, EV05162, EV05174, EV05178, EV05184, EV06018, EV06020, EV06026, EV06028, EV06036, EV06044, EV06046, EV06048, EV06056, EV06058, EV06062, EV06068, EV06072, EV06086, EV06088, EV06092, EV06094, EV06096, EV06098, EV06110, EV06128, EV06136, EV06140, EV06144, EV06148, EV06154, EV06162, EV06166, EV06178, EV06188, EV07038.

In another embodiment, the compound of formula (I) or (Ia) is set forth according to the following provisions:

A=O

R²═R³═R⁴ is H;

R¹ is OC(═O)OR⁵;

R⁵ is alkyl, alkenyl, or alkynyl; and

R⁷ is H, alkyl, alkenyl, or halogen.

In another embodiment, the compound of formula (I) or (Ia) has the following variable assignments:

A=O

R²═R³═R⁴ is H;

R¹ is OC(═O)OR⁵;

R⁵ is alkyl; and

R⁷ is H, alkyl, alkenyl, or halogen.

In another embodiment, the compound of formula (I) or (Ia) has the following variable assignments:

A=O

R²═R³═R⁴ is H;

R¹ is OC(═O)R⁵;

R⁵ is alkyl, alkyl ether, CCOC(═O)C, alkyl acetate, alkenyl, alkenyl acetate, alkynyl, or alkynyl acetate; and

R⁷ is H, alkyl, alkenyl, or halogen.

In another embodiment, the compound of formula (I) or (Ia) is set forth according to the following provisions:

A=O

R²═R³═R⁴ is H;

R¹ is C(═O)OR⁵;

R⁵ is alkyl, alkenyl, or alkynyl; and

R⁷ is H, alkyl, alkenyl, or halogen.

In another embodiment, the compound of formula (I) or (Ia) is set forth according to the following provisions:

A=O

R²═R³═R⁴ is H;

R¹ is OR⁵;

R⁵ is alkyl, alkenyl, or alkynyl; and

R⁷ is H, alkyl, alkenyl, or halogen.

In another embodiment, the compound of formula (I) or (Ia) is set forth according to the following provisions:

A=O

R¹═R³═R⁴ is H;

R² is OR⁵ or CR⁵;

R⁵ is alkyl, alkenyl, or alkynyl; and

R⁷ is H, alkyl, alkenyl, or halogen.

In another embodiment, the compound of formula (I) or (Ia) is set forth according to the following provisions:

A=O

R¹═R²═R⁴ is H;

R³ is OC(O)OR⁵;

R⁵ is alkyl, alkenyl, or alkynyl; and

R⁷ is H, alkyl, alkenyl, or halogen.

In another embodiment, the compound of formula (I) or (Ia) has the following variable assignments:

A=O

R¹═R²═R⁴ is H;

R³ is OC(═O)OR⁵;

R⁵ is alkyl; and

R⁷ is H, alkyl, alkenyl, or halogen.

In another embodiment, the compound of formula (I) or (Ia) has the following variable assignments:

A=O

R¹═R²═R⁴ is H;

R³ is OC(═O)R⁵;

R⁵ is alkyl, alkyl ether, CCOC(═O)C, alkyl acetate, alkenyl, alkenyl acetate, alkynyl, or alkynyl acetate; and

R⁷ is H, alkyl, alkenyl, or halogen,

In another embodiment, the compound of formula (I) or (Ia) is set forth according to the following provisions:

A=O

R¹═R²═R⁴ is H;

R³ is C(═O)OR⁵;

R⁵ is alkyl, alkenyl, or alkynyl; and

R⁷ is H, alkyl, alkenyl, or halogen.

In another embodiment, the compound of formula (I) or (Ia) is set forth according to the following provisions:

A=O

R¹═R²═R⁴ is H;

R³ is OR⁵;

R⁵ is alkyl, alkenyl, or alkynyl; and

R⁷ is H, alkyl, alkenyl, or halogen.

In another embodiment, the compound of formula (I) or (Ia) is set forth according to the following provisions:

A=O

R¹═R²═R³ is H;

R⁴ is OC(═O)OR⁵;

R⁵ is alkyl, alkenyl, or alkynyl; and

R⁷ is H, alkyl, alkenyl, or halogen.

In another embodiment, the compound of formula (I) or (Ia) has the following variable assignments:

A=O

R¹═R²═R³ is H;

R⁴ is OC(═O)OR⁵;

R⁵ is alkyl; and

R⁷ is H, alkyl, alkenyl, or halogen.

In another embodiment, the compound of formula (I) or (Ia) has the following variable assignments:

A=O

R¹═R²═R³ is H;

R⁴ is OC(═O)R⁵;

R⁵ is alkyl, alkyl ether, CCOC(═O)C, alkyl acetate, alkenyl, alkenyl acetate, alkynyl, or alkynyl acetate; and

R⁷ is H, alkyl, alkenyl, or halogen.

In another embodiment, the compound of formula (I) or (Ia) is set forth according to the following provisions:

A=O

R¹═R²═R³ is H;

R⁴ is C(═O)OR⁵;

R⁵ is alkyl, alkenyl, or alkynyl; and

R⁷ is H, alkyl, alkenyl, or halogen.

In another embodiment, the compound of formula (I) or (Ia) is set forth according to the following provisions:

A=O

R¹═R²═R³ is H;

R⁴ is OR⁵;

R⁵ is alkyl, alkenyl, or alkynyl; and

R⁷ is H, alkyl, alkenyl, or halogen.

In another embodiment, the compound of formula (I) or (Ia) is set forth according to the following provisions:

A=O

R²═R³═R⁴ is H;

R¹ is OCC(═O)N(R⁵)(R⁸) or CC(═O)N(R⁵)(R⁸);

R⁵ and R⁸ are independently selected from alkyl, alkenyl, or alkynyl; and

R⁷ is H, alkyl, alkenyl, or halogen,

In another embodiment, the compound of formula (I) or (Ia) is set forth according to the following provisions:

A=O

R²═R³═R⁴ is H;

R¹ is OCC(═O)N(R⁵)(R⁸) or CC(═O)N(R⁵)(R⁸);

R⁵ and R⁸ come together to form a C3-C10 ring which may be substituted by alkyl, alkenyl, or alkynyl; and

R⁷ is H, alkyl, alkenyl, or halogen.

In another embodiment, the invention provides an insect/pest repellent composition comprising compounds or formulations of the instant disclosure.

In an embodiment, the composition is in a form suitable for topical application to an animal.

In an embodiment, the invention provides a method for repelling pests comprising the step of applying a compound or composition of any of the disclosed embodiments to animals or a locus.

In another embodiment the animals are birds or mammals.

In another embodiment the mammals are humans, equines, felines, canines, bovines, or caprines.

In another embodiment the animals are equines or bovines.

In another embodiment the animals are humans.

Other obvious variations on the above-recited embodiments will be appreciated by persons skilled in the art.

For convenience, certain terms employed in the Specification, Examples, and appended Claims are collected here.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicates otherwise.

As used herein, the word “about”, where it is specifically used to describe a concentration, a mass, a weight, or a volume, is hereby defined to mean “plus or minus 10%” of the stated value.

The following abbreviations shall have the indicated meanings: AcOH=Acetic acid; PTSA p-Toluenesulphonic acid; DAST=Diethylaminosulphur trifluoride DCC=Dicyclohexylcarbodiimide; DCM=Dichloromethane; DEPT=Distortionless Enhancement by Polarisation Transfer; DIEA=Diisopropylethylamine; DMAP=Dimethylaminopyridine; DMF Dimethylformamide; DMSO=Dimethyl sulphsulphoxide EtOH=Ethanol; Eq=Equivalent; FDPP=Pentafluorophenyl diphenyl phosphinate; HOBt=Hydroxybenzotriazole, HPLC=High Pressure Liquid Chromatography; MeOH=Methanol; MS=Mass spectrometry; MS4A=Molecular sieves 4 Angstrom; MsCl Mesyl chloride; NBS═N-Bromosuccinimide; NCS═N-Chlorosuccinimide Pyr=Pyridine, Yld=Yield; Rf=Retardation factor; NMR=Nuclear Magnetic Resonance; XR═X-ray; t=Time; A.T.=Ambient temperature; TBAF=Tetrabutylammonium fluoride; TEA Triethylamine; THF=Tetrahydrofuran; Rt=Retention time TsCl=Tosyl chloride; TSA=Toluene sulphsulphonic acid; Vol.=Volume; COSY (Correlation Spectroscopy); ¹H-¹H homonuclear scalar coupling; NOESY (Nuclear Overhauser Effect Spectroscopy): ¹H-¹H homonuclear spatial coupling; HMBC (Heteronuclear Multiple Bond Correlation): ¹H-¹³C long-distance correlation; HSQC-TOCSY (Heteronuclear Multiple Quantum Correlation-Total Correlation Spectroscopy.

As used herein, the term “animal” includes all vertebrate animals including humans. It also includes an individual animal in all stages of development, including embryonic and fetal stages. In particular, the term “vertebrate animal” includes, but not limited to, humans, canines (e.g., dogs), felines (e.g., cats); equines (e.g., horses), bovines (e.g., cattle), ovine (e.g., sheep), porcine (e.g., pigs), as well as avians. The term “avian” as used herein refers to any species or subspecies of the taxonomic class ava, such as, but not limited to, chickens (breeders, broilers and layers), turkeys, ducks, a goose, a quail, pheasants, parrots, finches, hawks, crows and ratites including ostrich, emu and cassowary, and includes all avians kept as either companion or production animals.

As used herein, the term “aqueous suspension” includes mixtures of insoluble particles in water. Aqueous suspensions may contain the active material in admixture with excipients suitable for the manufacture of aqueous suspensions. Such excipients are suspending agents, for example, colloidal silica, sodium carboxymethylcellulose, methylcellulose, xanthan gum, hydroxy-propylmethylcellulose, sodium alginate, polvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide, with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents and/or bittering agents, such as those set forth above.

It will be understood by those of skill in the art that compounds of formula (I) may also be prepared by derivatization of other compounds (I) or by customary modifications of the synthesis routes described.

When the compounds of formula (I) contain suitably acidic or basic residues that enable the formation of veterinarily or pharmaceutically acceptable salts, the compounds may be reacted with suitable acids such as hydrochloric acid, hydrobromic acid, hydroiodic acid, sulfuric acid, phosphoric acid, formic acid, acetic acid, oxalic acid, benzene sulfonic acid, p-toluene sulfonic acid, dodecylbenzene sulfonic acid, methyl bromide, dimethyl sulfate or diethyl sulfate, and the like, typically at a temperature range of about −5° C. to about 150° C., preferably about 0 to about 20° C., in a suitable solvent.

Alternatively, compounds of formula (I) that contain acidic residues may be reacted with suitable bases, including organic amine bases or inorganic bases such as hydroxides, carbonates or bicarbonates of alkali metals or alkaline earth metals.

The formation of the salt is usually conducted in a dissolving or diluting agent. Suitable are e.g. aliphatic hydrocarbons as n-pentane, n-hexane or petrol ether, aromatic hydrocarbons, as toluene or xylenes, or ethers such as diethyl ether, methyl-tert.-butyl ether, tetrahydrofuran or dioxane, further ketones, as acetone, methyl-ethyl-ketone or methyl-isopropyl-ketone, as well as halogenated hydrocarbons as chlorobenzene, methylene chloride, ethylene chloride, chloroform or tetrachloroethylene. Also mixtures of those solvents can be used.

For the preparation of salts of compounds of formula (I) the compounds and salt forming agents are employed usually in a stoichiometric ratio. The excess of one or the other component can be useful.

If individual compounds cannot be prepared via the above-described routes, they can be prepared by derivatization of other compounds or by customary modifications of the synthesis routes described.

The reaction mixtures are typically worked up in a customary manner, for example by mixing a reaction product mixture containing an organic solvent with water, separating the phases, and, if appropriate, purifying the crude products by chromatography, for example on alumina or silica gel. If the intermediates and end products are obtained as solids, they may be purified by recrystallization or digestion.

### Animal Health Applications:

One important aspect of the invention is the use of the compounds of formula (I/Ia) or compositions comprising the compounds for the prevention of parasite infestation/infection in or on animals, accomplished via the repulsion of insect or other pest vectors. The compositions of the invention comprise a repellent effective amount of at least one compound of formula (I) in combination with a veterinarily acceptable carrier or diluent and optionally other non-active excipients. The compositions may be in a variety of solid and liquid forms which are suitable for various forms of application or administration to an animal. For example, the veterinary compositions comprising the inventive compounds may be in formulations suitable for oral administration, injectable administration, including subcutaneous and parenteral administration, and topical, pour-on, dermal or subdermal administration. The formulations are intended to be administered to an animal including, but is not limited to, mammals, birds and fish. Examples of mammals include but are not limited to humans, cattle, sheep, goats, llamas, alpacas, pigs, horses, donkeys, dogs, cats and other livestock or domestic mammals. Examples of birds include turkeys, chickens, ostriches and other livestock or domestic birds.

### Veterinary Compositions:

Topical, dermal and subdermal formulations may include, by way of non-limiting example, emulsions, creams, ointments, gels, pastes, powders, shampoos, pour-on formulations, ready-to-use formulations, spot-on solutions and suspensions, dips and sprays. Topical application of an inventive compound or of a composition including at least one inventive compound among active agent(s) therein, in the form of a spot-on, spray-on or pour-on composition, may allow for the inventive composition to be absorbed through the skin to achieve systemic levels, distributed through the sebaceous glands or on the surface of the skin achieving levels throughout the coat. When the compound is distributed through the sebaceous glands, they may act as a reservoir, whereby there may be a long-lasting effect (up to several months) effect. Spot-on formulations are typically applied in a localized region which refers to an area other than the entire animal. In one embodiment, the location may be between the shoulders. In another embodiment it may be a stripe, e.g. a stripe from head to tail of the animal.

Pour-on formulations are described in U.S. Pat. No. 6,010,710, also incorporated herein by reference. Pour-on formulations may be advantageously oily, and generally comprise a diluent or vehicle and also a solvent (e.g. an organic solvent) for the active ingredient if the latter is not soluble in the diluent.

Organic solvents that can be used in the invention include, but are not limited to, acetyltributyl citrate, fatty acid esters such as the dimethyl ester, diisobutyl adipate, acetone, acetonitrile, benzyl alcohol, ethyl alcohol, butyl diglycol, dimethylacetamide, dimethylformamide, dimethyl sulfoxide, dipropylene glycol n-butyl ether, ethanol, isopropanol, methanol, ethylene glycol monoethyl ether, ethylene glycol monomethyl ether, monomethylacetamide, dipropylene glycol monomethyl ether, liquid polyoxyethylene glycols, propylene glycol, 2-pyrrolidone (e.g. N-methylpyrrolidone), diethylene glycol monoethyl ether, ethylene glycol, triacetin, C₁-C₁₀ esters of carboxylic acids such as butyl or octyl acetate, and diethyl phthalate, or a mixture of at least two of these solvents.

The solvent will be used in proportion with the concentration of the active agent compound and its solubility in this solvent. It will be sought to have the lowest possible volume. The vehicle makes up the difference to 100%.

A vehicle or diluent for the formulations may include dimethyl sulfoxide (DMSO), glycol derivatives such as, for example, propylene glycol, glycol ethers, polyethylene glycols or glycerol. As vehicle or diluent, mention may also be made of plant oils such as, but not limited to soybean oil, groundnut oil, castor oil, corn oil, cotton oil, olive oil, grape seed oil, sunflower oil, etc.; mineral oils such as, but not limited to, petrolatum, paraffin, silicone, etc.; aliphatic or cyclic hydrocarbons or alternatively, for example, medium-chain (such as C₈ to C₁₂) triglycerides.

In another embodiment of the invention, an emollient and/or spreading and/or film-forming agent may be added. In one embodiment, the emollient and/or spreading and/or film-forming agent may be:

(a) polyvinylpyrrolidone, polyvinyl alcohols, copolymers of vinyl acetate and vinylpyrrolidone, polyethylene glycols, benzyl alcohol, mannitol, glycerol, sorbitol, polyoxyethylenated sorbitan esters; lecithin, sodium carboxymethylcellulose, silicone oils, polydiorganosiloxane oils (such as polydimethylsiloxane (PDMS) oils), for example those containing silanol functionalities, or a 45V2 oil,

(b) anionic surfactants such as alkaline stearates, sodium, potassium or ammonium stearates; calcium stearate, triethanolamine stearate; sodium abietate; alkyl sulphates (e.g. sodium lauryl sulphate and sodium cetyl sulphate); sodium dodecylbenzenesulphonate, sodium dioctylsulphosuccinate; fatty acids (e.g. those derived from coconut oil),

(c) cationic surfactants include water-soluble quaternary ammonium salts of formula N⁺R′R″R″R″″, Y⁻ in which the radicals R are optionally hydroxylated hydrocarbon radicals and Y⁻ is an anion of a strong acid such as the halide, sulphate and sulphonate anions; cetyltrimethylammonium bromide is among the cationic surfactants which can be used,

(d) amine salts of formula N⁺ HR′R″R′″ in which the radicals R are optionally hydroxylated hydrocarbon radicals; octadecylamine hydrochloride is among the cationic surfactants which can be used,

(e) nonionic surfactants such as sorbitan esters, which are optionally polyoxyethylenated (e.g. polysorbate 80), polyoxyethylenated alkyl ethers; polyoxypropylated fatty alcohols such as polyoxypropylene-styrol ether; polyethylene glycol stearate, polyoxyethylenated derivatives of castor oil, polyglycerol esters, polyoxyethylenated fatty alcohols, polyoxyethylenated fatty acids, copolymers of ethylene oxide and propylene oxide,

(f) amphoteric surfactants such as the substituted lauryl compounds of betaine; or

(g) a mixture of at least two of these agents.

In one embodiment of the amount of emollient, the emollient used may be in a proportion of from about 0.1 to 50% or 0.25 to 5%, by volume. In another embodiment, the emollient used may be in a proportion of from about 0.1% to about 30%, about 1% to about 30%, about 1% to about 20%, or about 5% to about 20% by volume.

In another embodiment of the invention, the composition may be in ready-to-use solution form as is described in U.S. Pat. No. 6,395,765, incorporated herein by reference. In addition to the compounds of the invention, the ready-to-use solution may contain a crystallization inhibitor and an organic solvent or a mixture of organic solvents. In some embodiments, water may be included with the organic solvent.

In various embodiments of the invention, the compositions may include a crystallization inhibitor in an amount of about 1 to about 50% (w/v) or about 5 to about 40% (w/v) based on the total weight of the formulation. In other embodiments, the amount of crystallization inhibitor in the inventive formulations may be about 1% to about 30%, about 5% to about 20%, about 1% to about 15%, or about 1% to about 10% (w/w). The type of crystallization inhibitor used in the inventive formulations is not limited as long as it functions to inhibit crystallization of the active or inactive agents from the formulation. For example, in certain embodiments of the invention, a solvent or co-solvent of the formulation may also function as a crystallization inhibitor if it sufficiently inhibits the formation of crystals from forming over time when the formulation is administered.

Crystallization inhibitors which are useful for the invention include, but are not limited to:

(a) polyvinylpyrrolidone, polyvinyl alcohols, copolymers of vinyl acetate and vinylpyrrolidone, polyethylene glycols, benzyl alcohol, dimethylformamide, dimethylacetamide, dimethylsulfoxide, 2-pyrrolidone, N-methylpyrrolidone, mannitol, glycerol, sorbitol or polyoxyethylenated esters of sorbitan; lecithin or sodium carboxymethylcellulose; or acrylic derivatives, such as acrylates or methacrylates or polymers or copolymers thereof, polyethyleneglycols (PEG) or polymers containing polyethyleneglycols, such as glycofurol and the like, and others;

(b) anionic surfactants, such as alkaline stearates (e.g. sodium, potassium or ammonium stearate); calcium stearate or triethanolamine stearate; sodium abietate; alkyl sulphates, which include but are not limited to sodium lauryl sulphate and sodium cetyl sulphate; sodium dodecylbenzenesulphonate or sodium dioctyl sulphosuccinate; or fatty acids (e.g. coconut oil);

(c) cationic surfactants, such as water-soluble quaternary ammonium salts of formula N⁺R′R″R′″R″″Y⁻, in which the R radicals are identical or different optionally hydroxylated hydrocarbon radicals and Y⁻ is an anion of a strong acid, such as halide, sulphate and sulphonate anions; cetyltrimethylammonium bromide is one of the cationic surfactants which can be used;

(d) amine salts of formula N⁺HR′R″R′″, in which the R radicals are identical or different optionally hydroxylated hydrocarbon radicals; octadecylamine hydrochloride is one of the cationic surfactants which can be used;

(e) non-ionic surfactants, such as optionally polyoxyethylenated esters of sorbitan, e.g. Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide;

(f) amphoteric surfactants, such as substituted lauryl compounds of betaine;

(g) a mixture of at least two of the compounds listed in (a)-(f) above; or

(h) an organic solvent or mixture of solvents which inhibit the formation of crystals or amorphous solid after the formulation is administered.

In one embodiment of the crystallization inhibitor, a crystallization inhibitor pair will be used. Such pairs include, for example, the combination of a film-forming agent of polymeric type and of a surface-active agent. These agents will be selected from the compounds mentioned above as crystallization inhibitor.

In some embodiments, the organic solvent(s) may have a dielectric constant of between about 10 and about 35 or between about 20 and about 30. In other embodiments, the organic solvent may have a dielectric constant of between about 10 and about 40 or between about 20 and about 30. The content of this organic solvent or mixture of solvents in the overall composition is not limited and will be present in an amount sufficient to dissolve the desired components to a desired concentration. As discussed above, the organic solvent may also function as a crystallization inhibitor in the formulation.

In some embodiments, one or more of the organic solvent(s) may have a boiling point of below about 100° C., or below about 80° C. In other embodiments, the organic solvent(s) may have a boiling point of below about 300° C., below about 250° C., below about 230° C., below about 210° C. or below about 200° C.

In some embodiments where there is a mixture of solvents, i.e. a solvent and a co-solvent, the solvents may be present in the composition in a weight/weight (W/W) ratio of about 1/50 to about 1/1. Typically the solvents will be in a ratio of about 1/30 to about 1/1, about 1/20 to about 1/1, or about 1/15 to about 1/1 by weight Preferably, the two solvents will be present in a weight/weight ratio of about 1/15 to about 1/2. In some embodiments, at least one of the solvents present may act as to improve solubility of the active agent or as a drying promoter. In particular embodiments, at least one of the solvents will be miscible with water.

The formulation may also comprise an antioxidizing agent intended to inhibit oxidation in air, this agent may be present in a proportion of about 0.005 to about 1% (w/v), about 0.01 to about 0.1%, or about 0.01 to about 0.05%.

In one embodiment of the film-forming agent, the agents are of the polymeric type which include but are not limited to the various grades of polyvinylpyrrolidone, polyvinyl alcohols, and copolymers of vinyl acetate and of vinylpyrrolidone.

In one embodiment of the surface-active agents, the agents include but are not limited to those made of non-ionic surfactants; in another embodiment of the surface active agents, the agent is a polyoxyethylenated esters of sorbitan and in yet another embodiment of the surface-active agent, the agents include the various grades of polysorbate, for example Polysorbate 80.

In another embodiment of the invention, the film-forming agent and the surface-active agent may be incorporated in similar or identical amounts within the limit of the total amounts of crystallization inhibitor mentioned elsewhere.

The crystallization inhibitor inhibits the formation of crystals on the coat, and improves the maintenance of the cosmetic appearance of the skin or fur; that is to say without a tendency towards sticking or towards a sticky appearance, despite the high concentration of active material. Substances other than those mentioned herein may be used as crystallization inhibitors in the present invention. In one embodiment, the effectiveness of the crystallization inhibitor may be demonstrated by a test according to which 0.3 mL of a solution comprising 10% (w/v) of the active agent in an appropriate solvent as defined above, and 10% (w/v) of the compound acting as a crystallization inhibitor are placed on a glass slide at 20° C. for 24 hours, after which fewer than 10 crystals, preferably 0 crystals, are seen with the naked eye on the glass slide.

In one embodiment of the antioxidizing agents, the agents are those conventional in the art and include but are not limited to butylated hydroxyanisole, butylated hydroxytoluene, ascorbic acid, sodium metabisulphite, propyl gallate, sodium thiosulphate or a mixture of at least two compounds with antioxidant properties.

The formulation adjuvants discussed above are well known to the practitioner in this art and may be obtained commercially or through known techniques. These concentrated compositions are generally prepared by simple mixing of the constituents as defined above; advantageously, the starting point is to mix the active material in the main solvent and then the other ingredients or adjuvants are added.

The volume of the formulation applied will depend on the type of animal and the size of the animal as well as the strength of the formulation and the potency of the active agents. In one embodiment, an amount of about 0.1 to about 20 ml of the formulation may be applied to the animal. In other embodiment for the volume, the volume may be about 0.1 to about 10 ml, about 0.1 to about 5 ml, about 0.5 ml to about 10 ml, or about 0.3 to about 3 ml.

In another embodiment of the invention, application of a spot-on formulation according to the present invention may also provide long-lasting and broad-spectrum efficacy when the solution is applied to the mammal or bird. The spot-on formulations provide for topical administration of a concentrated solution, suspension, microemulsion or emulsion for intermittent application to a spot on the animal, generally between the two shoulders (solution of spot-on type).

For spot-on formulations, the carrier may be a liquid carrier vehicle as described in U.S. Pat. No. 6,426,333 (incorporated herein by reference), which in one embodiment of the spot-on formulation may comprise a solvent or mixture of solvents including, but not limited to, acetone, an aliphatic alcohol such as methanol, ethanol, propanol, butanol, isopropanol, pentanol, hexanol, heptanol, octanol, nonanol, cyclopentanol, cyclohexanol, ethylene glycol, propylene glycol and the like; an aromatic alcohol such as phenol, cresol, naphthol, benzyl alcohol and the like; acetonitrile, butyl diglycol, an organic amide such as dimethylacetamide, dimethylformamide, monomethylacetamide, 2-pyrrolidone, N-methylpyrrolidone, vinylpyrrolidone and the like; dimethylsulfoxide (DMSO), a glycol polymer or an ether thereof, such as polyethylene glycol (PEG) of various grades, polypropylene glycols of various grades, dipropylene glycol n-butyl ether, ethylene glycol monoethyl ether, ethylene glycol monomethyl ether, dipropylene glycol monomethyl ether, diethylene glycol monoethyl ether, ethylene glycol, diethyl phthalate fatty acid esters, such as the diethyl ester or diisobutyl adipate, or a mixture of at least two of these solvents.

The liquid carrier vehicle may optionally contain a crystallization inhibitor including, but not limited to, those described in (a) to (h) above, or a compound that may act both as a solvent and a crystallization inhibitor (as defined above), or a mixture of these crystallization inhibitors.

Spot-on formulations may be prepared by dissolving the active ingredients into the pharmaceutically or veterinary acceptable vehicle. Alternatively, the spot-on formulation may be prepared by encapsulation of the active ingredient to leave a residue of the therapeutic agent on the surface of the animal. These formulations will vary with regard to the weight of the therapeutic agent in the combination depending on the species of host animal to be treated, the severity and type of infection and the body weight of the host.

Dosage forms may typically contain from about 0.1 mg to about 5 g. in other embodiments, the dosage form may contain about 0.5 mg to about 5 g of an active agent. In one embodiment of the dosage form, the dosage may contain from about 1 mg to about 500 mg of an active agent, typically about 25 mg, about 50 mg, about 100 mg, about 200 mg, about 300 mg, about 400 mg, about 500 mg, about 600 mg, about 800 mg, or about 1000 mg.

In one embodiment of the invention, the active agent may be present in the formulation at a concentration of about 0.05 to about 10% weight/volume. In another embodiment of the invention, the active agent may be present in the formulation as a concentration from about 0.1 to about 2% weight/volume. In yet another embodiment of the invention, the active agent may be present in the formulation as a concentration from about 0.25 to about 1.5% weight/volume. In still another embodiment of the invention, the active agent may be present in the formulation as a concentration about 1% weight/volume.

### Methods of Treatment:

As discussed above, the compounds of formula (I) or (Ia) are effective in repelling insects and pests, and therefore may prevent insect/pest-borne infestations in animals or humans. In one embodiment, the invention provides a method for repelling insects or other pests away from an animal, comprising administering a repellent effective amount of a compound of formula (I) or (Ia), or veterinarily or pharmaceutically acceptable salts thereof, or a composition comprising the compounds, to the animal.

In still another embodiment of the invention, a method is provided for repulsion of insects/pests at a locus, which comprises administering or applying a repellent effective amount of a compound of formula (I) or (Ia), or veterinarily acceptable salts thereof, to the locus. With respect to animal health applications, “locus” is intended to mean a habitat, breeding ground, area, material or environment in which a parasite is growing or may grow, including in or on an animal.

Mammals which can be treated include but are not limited to humans, cats, dogs, cattle, chickens, cows, deer, goats, horses, llamas, pigs, sheep and yaks. In one embodiment of the invention, the mammals treated are humans, cats or dogs.

When an anthelmintic agent is added to the composition of the invention, the composition can also be used to treat against endoparasites such as those helminths selected from the group consisting of Anaplocephala, Ancylostoma, Anecator, Ascaris, Capillaria, Cooperia, Dipylidium, Dirofilaria, Echinococcus, Enterobius, Fasciola, Haemonchus, Oesophagostumum, Ostertagia, Oxyuris spp, Toxocara, Strongyloides, Strongylus spp., Toxascaris, Trichinella, Trichuris, and Trichostrongylus. The inventive compounds are particularly effective against organisms from the class of Protozoa, for example, Eimeria spp. and Plasmodia spp.

In each aspect of the invention, the compounds and compositions of the invention can be applied against a single organism/parasite or combinations thereof.

Additional pharmaceutical, pesticidal or veterinary active ingredients, which include, but are not limited to, parasiticidals including acaricides, anthelmintics, endectocides and insecticides, may also be added to the compositions of the invention. Anti-parasitic agents may include both ectoparasiticidal and endoparasiticidal agents. Veterinary pharmaceutical agents are well-known in the art (see e.g. Plumb' Veterinary Drug Handbook, 5^{th} Edition, ed. Donald C. Plumb, Blackwell Publishing, (2005) or The Merck Veterinary Manual, 9^{th} Edition, (January 2005)) and include but are not limited to acarbose, acepromazine maleate, acetaminophen, acetazolamide, acetazolamide sodium, acetic acid, acetohydroxamic acid, acetylcysteine, acitretin, acyclovir, albendazole, albuterol sulfate, alfentanil, allopurinol, alprazolam, altrenogest, amantadine, amikacin sulfate, aminocaproic acid, aminopentamide hydrogen sulfate, aminophylline/theophylline, amiodarone, amitraz, amitriptyline, amlodipine besylate, ammonium chloride, ammonium molybdenate, amoxicillin, amoxicillin, clavulanate potassium, amphotericin B desoxycholate, amphotericin B ampicillin, amprolium, antacids (oral), antivenin, apomorphione, apramycin sulfate, ascorbic acid, asparaginase, aspiring, atenolol, atipamezole, atracurium besylate, atropine sulfate, aurnofin, aurothioglucose, azaperone, azathioprine, azithromycin, baclofen, barbituates, benazepril, betamethasone, bethanechol chloride, bisacodyl, bismuth subsalicylate, bleomycin sulfate, boldenone undecylenate, bromides, bromocriptine mesylate, budenoside, buprenorphine, buspirone, busulfan, butorphanol tartrate, cabergoline, calcitonin salmon, calcitrol, calcium salts, captopril, carbenicillin indanyl sodium, carbimazole, carboplatin, carnitine, carprofen, carvedilol, cefadroxil, cefazolin sodium, cefixime, cefoperazone sodium, cefotaxime sodium, cefotetan disodium, cefoxitin sodium, cefpodoxime proxetil, ceftazidime, ceftiofur sodium, ceftiofur, ceftiaxone sodium, cephalexin, cephalosporins, cephapirin, charcoal (activated), chlorambucil, chloramphenicol, chlordiazepoxide, chlordiazepoxide +/−clidinium bromide, chlorothiazide, chlorpheniramine maleate, chlorpromazine, chlorpropamide, chlortetracycline, chorionic gonadotropin (HCG), chromium, cimetidine, ciprofloxacin, cisapride, cisplatin, citrate salts, clarithromycin, clemastine fumarate, clenbuterol, clindamycin, clofazimine, clomipramine, claonazepam, clonidine, cloprostenol sodium, clorazepate dipotassium, clorsulon, cloxacillin, codeine phosphate, colchicine, corticotropin (ACTH), cosyntropin, cyclophosphamide, cyclosporine, cyproheptadine, cytarabine, dacarbazine, dactinomycin/actinomycin D, dalteparin sodium, danazol, dantrolene sodium, dapsone, decoquinate, deferoxamine mesylate, deracoxib, deslorelin acetate, desmopressin acetate, desoxycorticosterone pivalate, detomidine, dexamethasone, dexpanthenol, dexraazoxane, dextran, diazepam, diazoxide (oral), dichlorphenamide, dichlorvos, diclofenac sodium, dicloxacillin, diethylcarbamazine citrate, diethylstilbestrol (DES), difloxacin, digoxin, dihydrotachysterol (DHT), diltiazem, dimenhydrinate, dimercaprol/BAL, dimethyl sulfoxide, dinoprost tromethamine, diphenylhydramine, disopyramide phosphate, dobutamine, docusate/DSS, dolasetron mesylate, domperidone, dopamine, doramectin, doxapram, doxepin, doxorubicin, doxycycline, edetate calcium disodium.calcium EDTA, edrophonium chloride, enalapril/enalaprilat, enoxaparin sodium, enrofloxacin, ephedrine sulfate, epinephrine, epoetin/erythropoietin, eprinomectin, epsiprantel, erythromycin, esmolol, estradiol cypionate, ethacrynic acid/ethacrynate sodium, ethanol (alcohol), etidronate sodium, etodolac, etomidate, euthanasia agents w/pentobarbital, famotidine, fatty acids (essential/omega), felbamate, fenbendazole, fentanyl, ferrous sulfate, filgrastim, finasteride, fipronil, florfenicol, fluconazole, flucytosine, fludrocortisone acetate, flumazenil, flumethasone, flunixin meglumine, fluorouracil (5-FU), fluoxetine, fluticasone propionate, fluvoxamine maleate, fomepizole (4-MP), furazolidone, furosemide, gabapentin, gemcitabine, gentamicin sulfate, glimepiride, glipizide, glucagon, glucocorticoid agents, glucosamine/chondroitin sulfate, glutamine, glyburide, glycerine (oral), glycopyrrolate, gonadorelin, grisseofulvin, guaifenesin, halothane, hemoglobin glutamer-200 (OXYGLOBIN®), heparin, hetastarch, hyaluronate sodium, hydrazaline, hydrochlorothiazide, hydrocodone bitartrate, hydrocortisone, hydromorphone, hydroxyurea, hydroxyzine, ifosfamide, imidacloprid, imidocarb dipropinate, impenem-cilastatin sodium, imipramine, inamrinone lactate, insulin, interferon alfa-2a (human recombinant), iodide (sodium/potassium), ipecac (syrup), ipodate sodium, iron dextran, isoflurane, isoproterenol, isotretinoin, isoxsuprine, itraconazole, ivermectin, kaolin/pectin, ketamine, ketoconazole, ketoprofen, ketorolac tromethamine, lactulose, leuprolide, levamisole, levetiracetam, levothyroxine sodium, lidocaine, lincomycin, liothyronine sodium, lisinopril, lomustine (CCNU), lufenuron, lysine, magnesium, mannitol, marbofloxacin, mechlorethamine, meclizine, meclofenamic acid, medetomidine, medium chain triglycerides, medroxyprogesterone acetate, megestrol acetate, melarsomine, melatonin, meloxican, melphalan, meperidine, mercaptopurine, meropenem, metformin, methadone, methazolamide, methenamine mandelate/hippurate, methimazole, methionine, methocarbamol, methohexital sodium, methotrexate, methoxyflurane, methylene blue, methylphenidate, methylprednisolone, metoclopramide, metoprolol, metronidaxole, mexiletine, mibolerlone, midazolam milbemycin oxime, mineral oil, minocycline, misoprostol, mitotane, mitoxantrone, morantel tartrate, morphine sulfate, moxidectin, naloxone, mandrolone decanoate, naproxen, narcotic (opiate) agonist analgesics, neomycin sulfate, neostigmine, niacinamide, nitazoxanide, nitenpyram, nitrofurantoin, nitroglycerin, nitroprusside sodium, nizatidine, novobiocin sodium, nystatin, octreotide acetate, olsalazine sodium, omeprozole, ondansetron, opiate antidiarrheals, orbifloxacin, oxacillin sodium, oxazepam, oxfendazole, oxibutynin chloride, oxymorphone, oxytretracycline, oxytocin, pamidronate disodium, pancreplipase, pancuronium bromide, paromomycin sulfate, parozetine, pencillamine, penicillins including penicillin G and penicillin V potassium, pentazocine, pentobarbital sodium, pentosan polysulfate sodium, pentoxifylline, pergolide mesylate, phenobarbital, phenoxybenzamine, pheylbutazone, phenylephrine, phenypropanolamine, phenyloin sodium, pheromones, parenteral phosphate, phytonadione/vitamin pimobendan, piperazine, pirlimycin, piroxicam, polysulfated glycosaminoglycan, ponazuril, potassium chloride, pralidoxime chloride, praziquantel, prazosin, prednisolone/prednisone, primidone, procainamide, procarbazine, prochlorperazine, propantheline bromide, propionibacterium acnes injection, propofol, propranolol, protamine sulfate, pseudoephedrine, psyllium hydrophilic mucilloid, pyrantel pamoate, pyridostigmine bromide, pyrilamine maleate, pyrimethamine, quinacrine, quinidine, ranitidine, rifampin, s-adenosyl-methionine (SAMe), saline/hyperosmotic laxative, selamectin, selegiline/I-deprenyl, sertraline, sevelamer, sevoflurane, silymarin/milk thistle, sodium bicarbonate, sodium polystyrene sulfonate, sodium stibogluconate, sodium sulfate, sodum thiosulfate, somatotropin, sotalol, spectinomycin, spironolactone, stanozolol, streptokinase, streptozocin, succimer, succinylcholine chloride, sucralfate, sufentanil citrate, sulfachlorpyridazine sodium, sulfadiazine/trimethroprim, sulfamethoxazole/trimethoprim, sulfadimentoxine, sulfadimethoxine/ormetoprim, sulfasalazine, taurine, tepoxaline, terbinafline, terbutaline sulfate, testosterone, tetracycline, thiabendazole, thiacetarsamide sodium, thiamine, thioguanine, thiopental sodium, thiotepa, thyrotropin, tiamulin, ticarcilin disodium, tiletamine/zolazepam, tilmocsin, tiopronin, tobramycin sulfate, tocainide, tolazoline, telfenamic acid, topiramate, tramadol, trimcinolone acetonide, trientine, trilostane, trimepraxine tartrate w/prednisolone, tripelennamine, tylosln, urdosiol, valproic acid, vanadium, vancomycin, vasopressin, vecuronium bromide, verapamil, vinblastine sulfate, vincristine sulfate, vitamin E/selenium, warfarin sodium, xylazine, yohimbine, zafirlukast, zidovudine (AZT), zinc acetate/zinc sulfate, zonisamide and mixtures thereof.

In one embodiment of the invention, arylpyrazole compounds may be added to the compositions of the invention. Arylpyrazoles may include but are not limited to those described in U.S. Pat. Nos. 6,001,384, 6,010,710, 6,083,519, 6,096,329, 6,174,540, 6,685,954 and 6,998,131, all of which are hereby incorporated by reference in their entirety, —each assigned to Merial, Ltd., Duluth, Ga.). A particularly preferred arylpyrazole compound that may be combined with the compounds of the invention is fipronil (5-amino-1-[2,6-dichloro-4-(trifluoromethyl)phenyl]-4-(trifluoromethylsulfinyl)pyrazole-3-carbonitrile, CAS No. 120068-37-3).

In another embodiment of the invention, nodulisporic acid and its derivatives (a class of known acaricidal, anthelmintic, anti-parasitic and insecticidal agents) may be added to the compositions of the invention. These compounds are used to treat or prevent infections in humans and animals and are described, for example, in U.S. Pat. Nos. 5,399,582, 5,962,499, 6,221,894 and 6,399,786, all of which are hereby incorporated by reference in their entirety. The compositions may include one or more of the known nodulisporic acid derivatives in the art, including all stereoisomers, such as those described in the literature cited above.

In another embodiment, anthelmintic compounds of the amino acetonitrile class (AAD) of compounds such as monepantel (ZOLVIX) and the like may be added to the compositions of the invention. These compounds are described, for example, in WO 2004/024704; Sager at al., Veterinary Parasitology, 2009, 159, 49-54; Kaminsky at al., Nature vol. 452, 13 Mar. 2008, 176-181.

The compositions of the invention may also be combined with paraherquamide compounds and derivatives of these compounds, including derquantel (see Ostlind et al., Research in Veterinary Science, 1990, 48, 260-61; and Ostlind et al, Medical and Veterinary Entomology, 1997, 11, 407-408). The paraherquamide family of compounds are known class of compounds that include a spirodioxepino indole core with activity against certain parasites (see Tet. Lett. 1981, 22, 135; J. Antibiotics 1990, 43, 1380, and J. Antibiotics 1991, 44, 492). In addition, the structurally related marcfortine family of compounds, such as marcfortines A-C, are also known and may be combined with the formulations of the invention (see J. Chem. Soc.—Chem. Comm. 1980, 601 and Tet. Lett. 1981, 22, 1977). Further references to the paraherquamide derivatives can be found, for example, in WO 91/09961, WO 92/22555, WO 97/03988, WO 01/076370, WO 09/004,432, U.S. Pat. No. 5,703,078 and U.S. Pat. No. 5,750,695, all of which are hereby incorporated by reference in their entirety.

In another embodiment, the compositions of the invention may be combined with cyclo-depsipeptide anthelmintic compounds including emodepside (see Willson et al, Parasitology, January 2003, 126(Pt 1):79-86).

In another embodiment of the invention, the class of acaricides or insecticides known as insect growth regulators (IGRs) may also be added to the compositions of the invention. Compounds belonging to this group are well known to the practitioner and represent a wide range of different chemical classes. These compounds all act by interfering with the development or growth of the insect pests. Insect growth regulators are described, for example, in U.S. Pat. No. 3,748,356; U.S. Pat. No. 3,818,047; U.S. Pat. No. 4,225,598; U.S. Pat. No. 4,798,837; U.S. Pat. No. 4,751,225, EP 0 179 022 or GB 2 140 010 as well as U.S. Pat. Nos. 6,096,329 and 6,685,954, all of which are hereby incorporated by reference in their entirety. Examples of IGRs suitable for use may include but are not limited to methoprene, pyriproxyfen, hydroprene, cyromazine, fluazuron, lufenuron, novaluron, pyrethroids, formamidines and 1-(2,6-difluorobenzoyl)-3-(2-fluoro-4-(trifluoromethyl)phenylurea.

An anthelmintic agent that may be combined with the compositions of the invention may be a benzenedisulfonamide compound, which includes but is not limited to clorsulon; or a cestodal agent, which includes but is not limited to praziquantel, pyrantel or morantel.

In some embodiments, a parasiticidal agent that may be combined with the compositions of the invention may be a biologically active peptide or protein including, but not limited to, depsipeptides, which act at the neuromuscular junction by stimulating presynaptic receptors belonging to the secretin receptor family resulting in the paralysis and death of parasites. In one embodiment of the depsipeptide, the depsipeptide may be emodepside.

In other embodiments, an insecticidal agent that may be combined with the compositions of the invention may be a spinosyn (e.g. spinosad) or a substituted pyridylmethyl derivative compound such as imidacloprid. Agents of this class are described above, and for example, in U.S. Pat. No. 4,742,060 or in EP 0 892 060, both of which are hereby incorporated by reference in their entirety.

For endoparasites, parasiticides which may be combined include but are not limited to pyrantel, morantel, the benzimidazoles (including albendazole, cambendazole, thiabendazole, fenbendazole, febantel, oxfendazole, oxibendazole, triclabendazole, mebendazole and netobimin), levamisole, closantel, rafoxanide, nitroxynil, disophenol and paraherquamide, For ectoparasites, insecticides which may be combined also include but are not limited to pyrethoids, organophosphates and neonicotinoids such as imidacloprid, as well as compounds such as metaflumizone, amitraz and ryanodine receptor antagonists.

The compositions of the invention may also comprise an antiparasitic macrocyclic lactone compound in combination with the active compound of the invention. The macrocyclic lactones include, but are not limited to, avermectins, such as abamectin, dimadectin, doramectin, emamectin, eprinomectin, ivermectin, latidectin, lepimectin, selamectin, ML 1,694,554 and milbemycins, such as milbemectin, milbemycin D, moxidectin and nemadectin. Also included are the 5-oxo and 5-oxime derivatives of said avermectins and milbemycins. Examples of compositions comprising macrocyclic lactones include but are not limited to those described in U.S. Pat. Nos. 6,426,333; 6,482,425; 6,962,713 and 6,998,131, all of which are incorporated by reference in their entirety; —each assigned to Medal, Ltd., Duluth, Ga.

The macrocyclic lactone compounds are known in the art and can easily be obtained commercially or through synthesis techniques known in the art. Reference is made to the widely available technical and commercial literature. For avermectins, ivermectin and abamectin, reference may be made, for example, to the work “Ivermectin and Abamectin”, 1989, by M. H. Fischer and H. Mrozik, William C. Campbell, published by Springer Verlag., or Albers-Schönberg et al. (1981), “Avermectins Structure Determination”, J. Am. Chem. Soc., 103, 4216-4221. For doramectin, “Veterinary Parasitology”, vol. 49, No. 1, July 1993, 5-15 may be consulted. For milbemycins, reference may be made, inter alia, to Davies H. G. et al., 1986, “Avermectins and Milbemycins”, Nat. Prod. Rep., 3, 87-121, Mrozik H. et al., 1983, Synthesis of Milbemycins from Avermectins, Tetrahedron Lett., 24, 5333-5336, U.S. Pat. No. 4,134,973 and EP 0 677 054.

Macrocyclic lactones are either natural products or are semi-synthetic derivatives thereof. The structure of the avermectins and milbemycins are closely related, e.g., by sharing a complex 16-membered macrocyclic lactone ring. The natural product avermectins are disclosed in U.S. Pat. No. 4,310,519 and the 22,23-dihydro avermectin compounds are disclosed in U.S. Pat. No. 4,199,569, each of which is incorporated herein by reference. Mention is also made of U.S. Pat. Nos. 4,468,390, 5,824,653, EP 0 007 812 A1, U.K. Patent Specification 1 390 336, EP 0 002 916, and New Zealand Patent No. 237 086, inter cilia, all of which are incorporated by reference in their entirety. Naturally occurring milbemycins are described in U.S. Pat. No. 3,950,360 as well as in the various references cited in “The Merck Index” 12 S. Budavari, Ed., Merck & Co., Inc. Whitehouse Station, New Jersey (1996). Latidectin is described in the “International Nonproprietary Names for Pharmaceutical Substances (INN)”, WHO Drug Information, vol. 17, no. 4, pp. 263-286, (2003). Semisynthetic derivatives of these classes of compounds are well known in the art and are described, for example, in U.S. Pat. Nos. 5,077,308, 4,859,657, 4,963,582, 4,855,317, 4,871,719, 4,874,749, 4,427,663, 4,310,519, 4,199,569, 5,055,596, 4,973,711, 4,978,677, 4,920,148 and EP 0 667 054, all of which are incorporated by reference in their entirety.

In yet another embodiment of the invention, adulticide insecticides and acaricides can also be added to the composition of the invention. These include pyrethrins (which include cinerin I, cinerin II, jasmolin I, jasmolin II, pyrethrin I, pyrethrin II and mixtures thereof) and pyrethroids, organophosphate (which included but are not limited to chlorfenvinphos, crotoxyphos, dichlorvos, heptenophos, mevinphos, monocrotophos, naled, TEPP, tetrachlorvinphos) and carbamates (which include but are not limited to benomyl, carbanolate, carbaryl, carbofuran, meththiocarb, metolcarb, promacyl, propoxur, aldicarb, butocarbaxim, oxamyl, thiocarboxime and thiofanox).

In addition to the other active agents mentioned above, combinations of two or more active agents may be used with the compounds of the invention in a composition to treat a desired spectrum of pests and parasites. It would be well within the skill level of the practitioner to decide which individual compound can be used in the inventive formulation to treat a particular infection of an insect.

### REFERENCES

Anna Petherick (2008-03-13). “How DEET jams insects' smell sensors”. Nature News. http://www.nature.com/news/2008/080313/full/news.2008.672.html. Retrieved 2008-03-16.
Mathias Ditzen, Maurizio Pellegrino, Leslie B. Vosshall (2008). “Insect Odorant Receptors Are Molecular Targets of the insect Repellent DEET”. Sciencexpress 319 (5871): 1838. doi:10.1126/science.1153121. PMID 18339904.
Syed, Z.; Leal, W S (2008). “Mosquitoes smell and avoid the insect repellent DEET”. Proc. Natl. Acad. Sci, USA 105 (36): 13598. doi:10.1073/pnas.0805312105,
Matsuda, Brent M.; Surgeoner, Gordon A.; Heal, James D.; Tucker, Arthur O.; Maciarello, Michael J. (1996). “Essential oil analysis and field evaluation of the citrosa plant “Pelargonium citrosum” as a repellent against populations of Aedes mosquitoes”. Journal of the American Mosquito Control Association 12 (1): 69-74. PMID 8723261.
University of North Carolina (3 Jul. 2002). “Independent study: DEET products superior for fending off mosquito bites”. Press release. http://www.unc.edu/news/archives/jul02/fradin070202.htm.
“Protection against Mosquitoes, Ticks, Fleas and Other Insects and Arthropods”. Travelers'Health—Yellow Book. Centers for Disease Control and Prevention. 2009-02-05. http://wwwn.cdc.gov/travel/yellowBookCh2-InsectsArthropods.aspx.
“Insect Repellent Use and Safety”. West Nile Virus. Centers for Disease Control and Prevention. 2007-01-12. http://www.cdc.gov/ncidod/dvbid/westnile/qa/insect_repellent.htm.
CDC. “Insect Repellent Use and Safety”. West Nile Virus. Centers for Disease Control and Prevention. 2007-01-12. http://www.cdc.gov/ncidod/dvbid/westnile/qa/insect_repellent.htm.
“Reregistration Eligibility Decision: DEET.” U.S. Environmental Protection Agency, Office of Prevention, Pesticides, and Toxic Substances. September 1998. pp 39-40
“DEET”. Pesticide Information Profile. EXTOXNET. October 1997. http://pmep.cce.cornell.edu/profiles/extoxnet/carbaryl-dicrotophos/deet-ext.html. Retrieved 2007-09-26.
“Insect Repellents”. Healthy Living. Health Canada. August 2009. http://www.hc-sc.gc.ca/hl-vs/iyh-vsv/life-vie/insect-eng.php. Retrieved 2010-07-09.
“Re-evaluation Decision Document: Personal insect repellents containing DEET (N,N-diethyl-m-toluamide and related compounds)”. Consumer Product Safety. Health Canada, 2002-04-15. http://www.hc-sc.gc.ca/cps-spc/pubs/pest/_decisions/rrd2002-01/index-eng.php. Retrieved 2010-07-09.
Corbel et al.; Stankiewicz, M; Pennetier, C; Fournier, D; Stojan, J; Girard, E; Dimitrov, M; Molgó, J et al. (2009). “Evidence for inhibition of cholinesterases in insect and mammalian nervous systems by the insect repellent deet”. BMC Biology 7:47 7:47. doi:10.1186/1741-7007-7-47. PMC 2739159. PMID 19656357. http://www.biomedcentral.com/1741-7007/7/47.
Purves, Dale, George J. Augustine, David Fitzpatrick, William C. Hall, Anthony-Samuel LaMantia, James O. McNamara, and Leonard E. White (2008). Neuroscience. 4th ed. Sinauer Associates. pp. 121-2. ISBN 978-0-87893-697-7.
Moss (1996). “Synergism of Toxicity of N,N-Diethyl-m-toluamide to German Cockroaches (Othoptera: Blattellidae) by Hydrolytic Enzyme Inhibitors.”. J. Econ. Entomol. 89 (5): 1151-1155. PMID 17450648.
U.S. Environmental Protection Agency. 1980. Office of Pesticides and Toxic Substances. N,N-diethyl-m-toluamide (Deet) Pesticide Registration Standard. December, 1980. 83 pp.
Mathai, A T; Pillai, K S; Deshmukh, P B (1989). “Acute toxicity of deet to a freshwater fish, Tilapia mossambica: Effect on tissue glutathione levels”. Journal of Environmental Biology 10 (2): 87-91. http://md1.csa.com/partners/viewrecord.php?requester=gs&collection=ENV&recid=2427671.&q=DEET+fish+-permethrin&uid=791055180&setcookie=yes.
J. Seo, V. G. Lee, S. D. Kim, C. J. Cha, J. H. Ahn and H. G. Hur (2005). “Biodegradation of the Insecticide N,N-Diethyl-m-Toluamide by Fungi: Identification and Toxicity of Metabolites”. Archives of Environmental Contamination and Toxicology 48 (3): 323-328. doi:10.1007/s00244-004-0029-9. PMID 15750774.
“Errol Zeiger, Raymond Tice, Brigette Brevard, (1999) N,N-Diethyl-m-toluamide (DEET) [134-62-3]-Review of Toxicological Literature” (PDF). http://ntp.niehs.nih.gov/ntp/htdocs/Chem_Background/ExSurnPdf/DEET.pdf. Retrieved July 20.
M. S. Fradin. Mosquitoes and Mosquito Repellents: A Clinician's Guide. Annals of Int. Med., 128(11):931-940, 1998.
T. Katz, J. H. Miller, and A. Hebert. Insect repellents: Historical perspectives and new developments. J. of the Am. Ac. of Derm., 58(5): 865-871, 2008.
A. R. Katritzky, Z. Wang, and S. Slavov. Synthesis and bioassay of improved mosquito repellents predicted from chemical structure. Proceedings of the National Academy of Sciences USA, 105(21):7359-64, 2008.
L. S, Nerio, J. Olivero-Verbel, and E. Stashenko. Repellent activity of essential oils: A review. Bioresource Tech., 101(1):372-378, 2010.
Z. Wang, J. Song, J. Chen, Z. Song, S. Shang, Z. Jiang, and Z. Han. Qsar study of mosquito repellents from terpenoid with a six-member-ring. Bioorganic & Med, Chem. Letters, 18(9):2854-2859, 2008.
M. Kramer, M. F. Feldlaufer, and K. R. Chauhan. Mosquito biting behavior: statistical power and sources of variation in toxicity and repellent bioassays. J. of Med. Entomology, 47(2):199-204, 2010.
C. F. Curtis. Fact and fiction in mosquito attraction and repulsion. Parasitology Today, 2(11):316-318, 1986.
R. Glatz and K. Bailey-Hill. Mimicking nature's noses: From receptor deorphaning to olfactory biosensing. Prog. in Neurobio., 93(2):270-296, 2011.
M. V. S. Suryanarayana, K. S. Pandey, S. Prakash, C. D. Raghuveeran, R. S. Dangi, R. V. Swamy, and K. M. Rao. Structure-activity relationship studies with mosquito repellent amides. J. of Pharma. Sciences, 80(11):1055-1057, 1991,
D. Ma, A. K. Bhattacharjee, R. Gupta, and J M. Karle. Predicting mosquito repellent potency of n,n-diethyl-m-toluamide (deet) analogs from molecular electronic properties. Am J Trop Med Hyg, 60(1):1-6, 1999.
W. G. Taylor, T. W. Hall, and C. E. Schreck. Synthesis and mosquito repellent properties of 2,2 dialkyl- and 2-alkyl-4,4-dimethyl-n-acetyloxazolidines. Pesticide Science, 46(41:307-314, 1996.
A. Katritzky, Z. Wang, and S. Slavov. Synthesis and bioassay of improved mosquito repellents predicted from chemical structure. Proceedings of the National Academy of Sciences, 105(21):7359-7364, 2008.
A. Katritzky. Codessa Pro software, 2002.
A. K. Bhattacharjee, W Dheranetra, D. A. Nichols, and R. Gupta. 3D pharmacophore model for insect repellent activity and discovery of new repellent candidates. QSAR & Combinatorial Science, 24(5):593-602, 2005.
Accelrys. Catalyst. software Inc. San Diego, Calif.
R. K. Gupta, A. K. Bhattacharjee, and D. M. Lee. Arthropods repellent pharmacophore models, compounds identified as fitting the pharmacophore models, and methods of making and using thereof, U.S. Pat. No. 7,897,162 B2, 2011.
R. Todeschini and V. Consoni. Handbook of Molecular Descriptors. Wiley-VCH, Torino, Italy, 2000.
Accelrys. DiscoveryStudio, San Diego, Calif. 2008.,
“ChemSpider.” from http://www.chemspider.com, 2007.
EPA. Estimation Programs Interface Suite™. v. 4.10. Washington, D.C., USA., United States Environmental Protection Agency, 2011.
A. P. Bradley. The use of the area under the ROC curve in the evaluation of machine learning algorithms, Pattern Recognition, 30(7) 1145-1159, 1997.
R. Kristam, V. J. Gillet, and R. A. Lewis. Comparison of conformational analysis techniques to generate pharmacophore hypotheses using catalyst, J. of Chem. Inf. Model, 2005, 45 (2):461-476.
Addinsoft. XLstat. 2011.
I. H. Witten, F. Eibe, and Mark A. Hall. Data Mining: Practical Machine Learning Tools and Techniques, Elsevier, Waikato, New Zealand, 2011.

The invention will now be further described by way of the following non-limiting examples.

### EXAMPLES

The following examples are provided to illustrate certain embodiments of the invention and are not to be construed in any way as limiting the scope of the invention.

### Example 1

### Modeling the “EV” Series of Coumarin Derivatives

Each of the molecules presented in Table 2 have been disclosed in USSN 61,501,485, and were subjected to the following processing steps, in preparation for developing the repellency model:
a. Two dimensional structure generation using Isis Draw;
b. Conversion of the file format in mol2 using DiscoveryStudio [17]. At this point every molecule has an unminimized three-dimensional structure;
c. Conformation analysis of all the molecules with a simulated annealing performed with the software Ampac, (the annealing principle and the parameters used in the calculation are described in Annex B). For the rest of the study, only the conformation with the lowest energy that comes out of the annealing conformational analysis were considered;
d. Every structure has to be checked for eventual incoherence (inadvertently generated during the annealing). i.e. transformation of an aromatic double bond in a single bond;
e. An energy minimization and atomic charges calculation were performed with Gaussian software (with and without a geometry optimization) at the HF/6-31G* level of calculation.
f. Once the minimal energy was calculated, the charge extraction was performed with the “antechamber” program from Amber tools and included into a moil file in order to allow the computation of molecular descriptors.

TABLE 2
“EV” compounds used in producing the repellency models

| | Repellency | |
|---|---|---|
| Name | Index | Structure |
| EV 04016 | 0.11 | 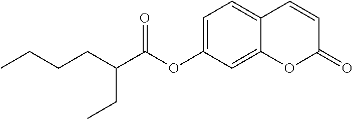 [SMILES: CCCCC(CC)C(=O)Oc1ccc2ccc(=O)oc2c1] |
| EV 04024 | 0.26 | 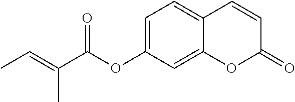 [SMILES: C/C=C(\C)C(=O)Oc1ccc2ccc(=O)oc2c1] |
| EV 04030 | −0.10 | 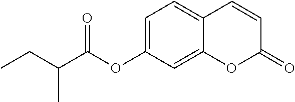 [SMILES: CCC(C)C(=O)Oc1ccc2ccc(=O)oc2c1] |
| EV 04032 | 0.19 | 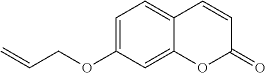 |
| EV 04036 | −0.06 | 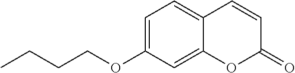 [SMILES: CCCCOc1ccc2ccc(=O)oc2c1] |
| EV 04042 | 0.43 | 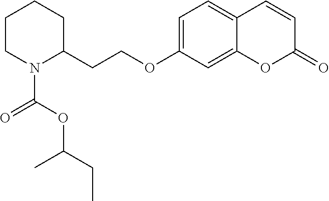 [SMILES: CCC(C)OC(=O)N1CCCCC1CCOc1ccc2ccc(=O)oc2c1] |
| EV 04054 | 0.66 | 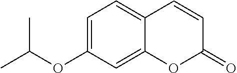 |
| EV 04058 | 0.76 | 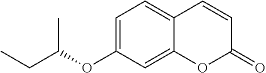 |
| EV 04062 | 0.04 | 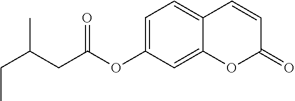 [SMILES: CCC(C)CC(=O)Oc1ccc2ccc(=O)oc2c1] |
| EV 04070 | 0.83 | 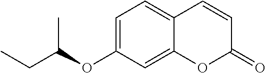 |
| EV 04084 | −0.01 | 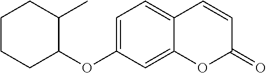 [SMILES: CC1CCCCC1Oc1ccc2ccc(=O)oc2c1] |
| EV 04090 | 0.40 | 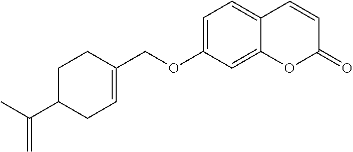 [SMILES: C=C(C)C1CC=C(COc2ccc3ccc(=O)oc3c2)CC1] |
| EV 04094 | 0.73 | 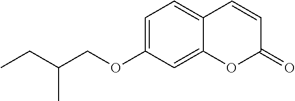 [SMILES: CCC(C)COc1ccc2ccc(=O)oc2c1] |
| EV 04114 | 0.86 | 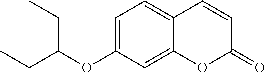 [SMILES: CCC(CC)Oc1ccc2ccc(=O)oc2c1] |
| EV 04122 | −0.36 | 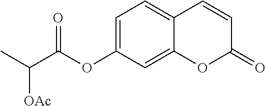 [SMILES: CC(=O)OC(C)C(=O)Oc1ccc2ccc(=O)oc2c1] |
| EV 04188 | 0.65 | 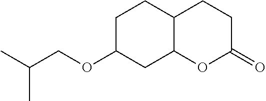 [SMILES: CC(C)COC1CCC2CCC(=O)OC2C1] |
| EV 05056 | 0.40 | 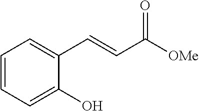 [SMILES: COC(=O)/C=C/c1ccccc1O] |
| EV 05084 | 0.30 | 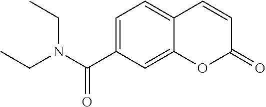 [SMILES: CCN(CC)C(=O)c1ccc2ccc(=O)oc2c1] |
| EV 05088 | 0.07 | 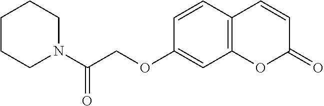 [SMILES: O=C(COc1ccc2ccc(=O)oc2c1)N1CCCCC1] |
| EV 05096 | 0.80 | 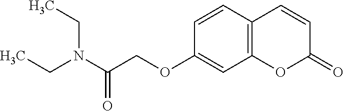 [SMILES: CCN(CC)C(=O)COc1ccc2ccc(=O)oc2c1] |
| EV 05118 | −0.10 | 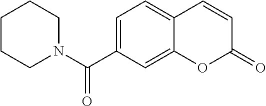 [SMILES: O=C(c1ccc2ccc(=O)oc2c1)N1CCCCC1] |
| EV 05120 | 0.71 | 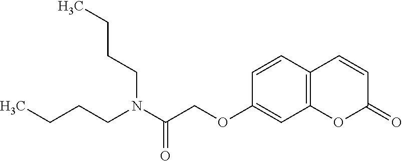 [SMILES: CCCCN(CCCC)C(=O)COc1ccc2ccc(=O)oc2c1] |
| EV 05134 | 0.78 | 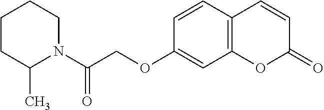 [SMILES: CC1CCCCN1C(=O)COc1ccc2ccc(=O)oc2c1] |
| EV 05138 | 0.72 | 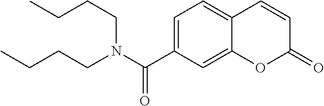 [SMILES: CCCCN(CCCC)C(=O)c1ccc2ccc(=O)oc2c1] |
| EV 05144 | 0.60 | 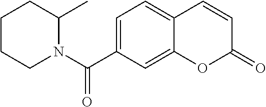 [SMILES: CC1CCCCN1C(=O)c1ccc2ccc(=O)oc2c1] |
| EV 05162 | 0.62 | 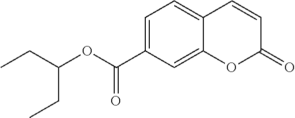 [SMILES: CCC(CC)OC(=O)c1ccc2ccc(=O)oc2c1] |
| EV 05174 | 0.69 | 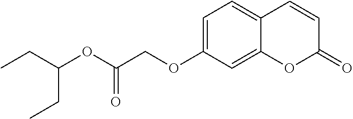 [SMILES: CCC(CC)OC(=O)COc1ccc2ccc(=O)oc2c1] |
| EV 05178 | 0.71 | 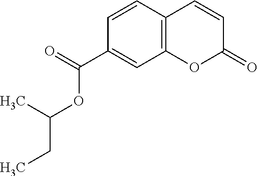 [SMILES: CCC(C)OC(=O)c1ccc2ccc(=O)oc2c1] |
| EV 06018 | 0.43 | 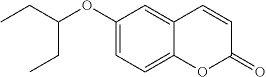 [SMILES: CCC(CC)Oc1ccc2oc(=O)ccc2c1] |
| EV 06026 | 0.88 | 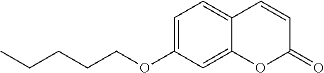 [SMILES: CCCCCOc1ccc2ccc(=O)oc2c1] |
| EV 06028 | 0.88 | 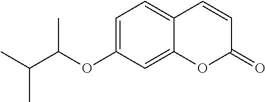 [SMILES: CC(C)C(C)Oc1ccc2ccc(=O)oc2c1] |
| EV 06036 | 0.73 | 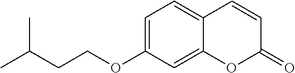 [SMILES: CC(C)CCOc1ccc2ccc(=O)oc2c1] |
| EV 06044 | 0.92 | 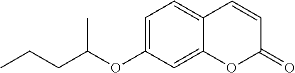 [SMILES: CCCC(C)Oc1ccc2ccc(=O)oc2c1] |
| EV 06046 | 0.08 | 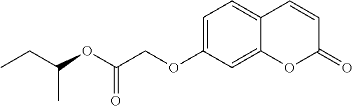 |
| EV 06058 | 0.52 | 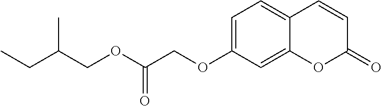 [SMILES: CCC(C)COC(=O)COc1ccc2ccc(=O)oc2c1] |
| EV 06062 | 0.97 | 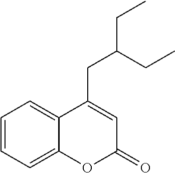 [SMILES: CCC(CC)Cc1cc(=O)oc2ccccc12] |
| EV 06068 | 0.88 | 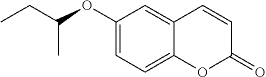 |
| EV 06088 | 0.82 | 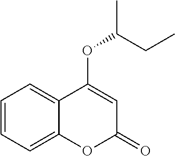 |
| EV 06092 | 0.35 | 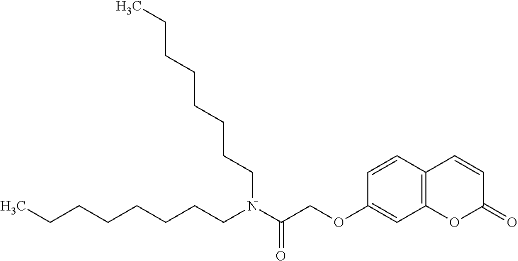 [SMILES: CCCCCCCCN(CCCCCCCC)C(=O)COc1ccc2ccc(=O)oc2c1] |
| EV 06096 | 0.91 | 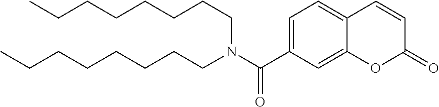 [SMILES: CCCCCCCCN(CCCCCCCC)C(=O)c1ccc2ccc(=O)oc2c1] |
| EV 06098 | 0.95 | 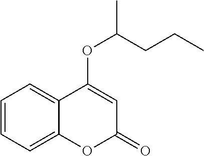 [SMILES: CCCC(C)Oc1cc(=O)oc2ccccc12] |
| EV 06110 | 0.78 | 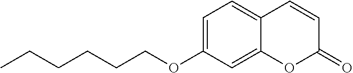 [SMILES: CCCCCCOc1ccc2ccc(=O)oc2c1] |
| EV 06128 | 0.34 | 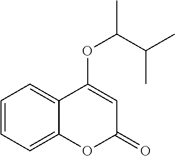 [SMILES: CC(C)C(C)Oc1cc(=O)oc2ccccc12] |
| EV 06136 | 0.77 | 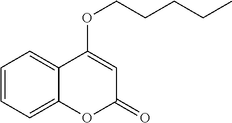 [SMILES: CCCCCOc1cc(=O)oc2ccccc12] |
| EV 06140 | 0.84 | 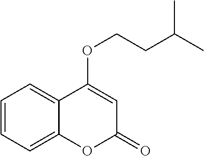 [SMILES: CC(C)CCOc1cc(=O)oc2ccccc12] |
| EV 06144 | 0.83 | 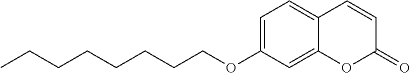 [SMILES: CCCCCCCCOc1ccc2ccc(=O)oc2c1] |
| EV 06148 | 0.92 | 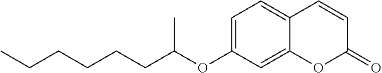 [SMILES: CCCCCCC(C)Oc1ccc2ccc(=O)oc2c1] |
| EV 06154 | 0.93 | 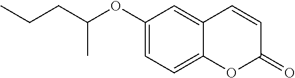 [SMILES: CCCC(C)Oc1ccc2oc(=O)ccc2c1] |
| EV 06162 | −0.55 | 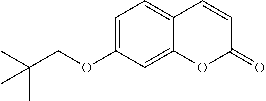 [SMILES: CC(C)(C)COc1ccc2ccc(=O)oc2c1] |
| EV 06166 | 0.25 | 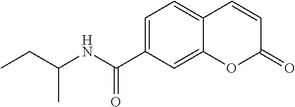 [SMILES: CCC(C)NC(=O)c1ccc2ccc(=O)oc2c1] |
| EV 06178 | 0.25 | 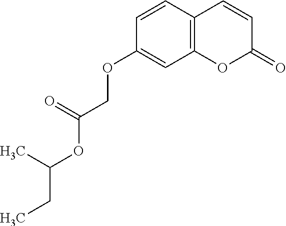 [SMILES: CCC(C)OC(=O)COc1ccc2ccc(=O)oc2c1] |
| EV 06188 | 0.87 | 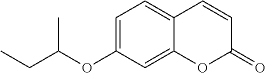 [SMILES: CCC(C)Oc1ccc2ccc(=O)oc2c1] |
| EV 07068 | 0.98 | 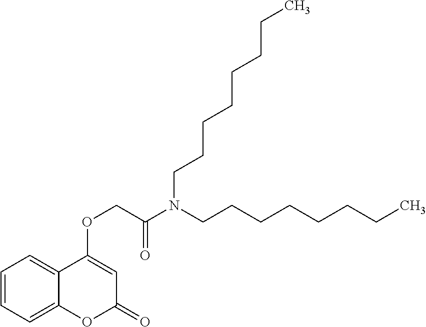 [SMILES: CCCCCCCCN(CCCCCCCC)C(=O)COc1cc(=O)oc2ccccc12] |
| EV07070 | 0.94 | 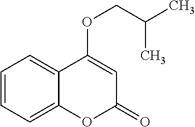 [SMILES: CC(C)COc1cc(=O)oc2ccccc12] |
| EV07072 | 0.98 | 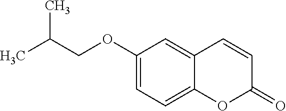 [SMILES: CC(C)COc1ccc2oc(=O)ccc2c1] |
| EV07078 | 0.91 | 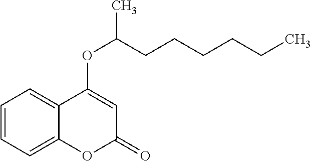 [SMILES: CCCCCCC(C)Oc1cc(=O)oc2ccccc12] |

DISCOVERYSTUDIO, which is a molecular modeling software platform used in drug design, was used for pharmacophore/QSAR modeling. It allows for the 3D visualization of molecules, and has the ability to calculate a large range of molecular descriptors. Also used were CHEMSPIDER, a website that provides both calculated and experimental values of molecular descriptors, and the software package, EPISUITE, which allows the calculation of molecular descriptors such as vapor pressure, and provides a source of experimental values.

Visual observation of the “EV” molecules suggested that the repellency index and the position of the lateral chain were important. Therefore, seven (7) new descriptors were created in order to reflect the chemical nature, the length and the position of the side chains.

TABLE 3
Molecular descriptors used to produce the repellency models

| | |
|---|---|
| Steric descriptors | 1. Shadow XY index |
| | 2. Shadow XZ index |
| | 3. Shadow YZ index |
| Physicochermical properties | 4. melting point |
| | 5. flash point |
| | 6. surface tension |
| | 7. density |
| | 8. Parachor index |
| | 9. molecular mass |
| | 10. molecular solubility |
| | 11. molecular volume |
| | 12. principal moment of inertia |
| Topological Descriptors | 13. N + O count |
| | 14. number of rotatable bonds |
| | 15. number of aromatic bonds |
| | 16. number of rings |
| | 17. number of aromatic rings |
| | 18. number of H acceptors |
| | 19. number of H donors |
| | 20. CHI_0 index |
| | 21. CHI_1 index |
| | 22. CHI_2 index |
| | 23. $Wiener index ( W = \frac{1}{2} {\sum }_{i = 1}^{N} {\sum }_{j = 1}^{N} {d}_{i , j} )$ |
| | 24. $Zagreb index ( Z = \frac{1}{2} {\sum }_{i = 1}^{N} {\sum }_{j = i + 1}^{M} {δ}_{i} {δ}_{j} )$ |
| | 25. substituent in position 4 on coumarin |
| | 26. substituent in position 6 on coumarin |
| | 27. substituent in position 7 on coumarin |
| | 28. lateral chain length |
| | 29. presence of an isopropyloxy group |
| | (O iso prop) |
| | 30. presence of an propyloxy group (O n prop) |
| | 31. tertiary amine |
| Lipophilic descriptors | 32. log P |
| Electronic descriptors | 33. E_{HOMO} (Energy of the Highest |
| | Occupied Molecular Orbital) |
| | 34. E_{LUMO} (Energy of the Lowest |
| | Unoccupied Molecular Orbital) |
| | 35. ΔH_{vaporization} |
| | 36. polarizability |
| | 37. molecular fractional polar surface area |
| | 38. molecular fractional polar surface area |
| | solvent accessible |
| | 39. dipole moment |

Descriptors 25 to 31 were designed in the laboratory for the special needs of the study. Descriptors 25 to 28 were designed to be applied on the coumarin scaffold only.

Linear and Classification Models.

Many computational techniques were used to elaborate QSAR models. Most of them can be classified in two families: classification and regression. The major difference between them is the way the property values are represented. Usually the regression methods model continuous values, whereas classification methods allow for modeling of discrete and/or nominal values (e.g., warm/temperate/cold; active/inactive). From a mathematical perspective, regression methods are based on the assumption that it is possible “to explain” a dependant variable (a property or an activity) with one or several explicative variable(s) called molecular descriptors (in computational chemistry). The results of a generated model are presented in the form of an equation with the property or the activity that has to be modeled on one side, and a weighted sum of descriptors on the other side. In this study, we only looked for linear relations described in Eq. 3, where βᵢ are coefficients and xᵢ are descriptors.

yᵢ=β₀+Σᵢ₌₁ⁿβᵢχᵢ (Eq.3)

The different algorithms used in classifications have the same goal: to separate the different instances depending on the values of their descriptors, in order to group them according to their activity value (supervised learning). As two different subsets of data are used (i.e., training and test sets) the two main coefficients that have to be taken into account, are: 1) the regression coefficient R², which is the measure of the ability of a QSAR model to reproduce the internal data in the training (goodness of fit), but which does not explain its robustness and predictive power; and 2) the prediction coefficient Q², which reflects the predictive power of a model.

The determination coefficient (R²) is the ratio of the regression sum of squares and the total sum of squares. The numerator takes into account the residual values coming from the regression whereas the denominator deals with the experimental values (Eq.4). R² is an indicator of fit to the regression line. The only difference in the calculation of Q² is that it only gives credit to molecules of the test set, which did not participate in the creation of the model.

$${R}^{2} = \frac{{{Σ}_{i} ( {\overline{y}}_{exp} - {y}_{ipred} )}^{2}}{{{Σ}_{i} ( {y}_{iexp} - {\overline{y}}_{exp} )}^{2}} \qquad ( Eq . 4 )$$

For every generated model, an internal validation was performed, each time following the principle of the “leave one out” protocol. This method predicts the activity from all the molecules from the training set with a model that has been generated on all the other molecules. Performing this for every instance in the dataset, a coefficient was calculated (R²_{LOO}).

For the classification models, there are no numerical values to deal with (they are replaced by nominal or binary values). In order to be able to analyze the results and to judge the relevance of those models, two tools were used: the number of correctly classified instances (percentage) and area under the ROC (Receiving Operating Characteristics) curve (AP Bradley, 1997). The ROC curves are obtained by putting each instance of the dataset on a graph, depending on the rates of true and false positives (strongly dependent of the order of the molecules in the database). The maximum value of the area under the ROC curve is 1. Furthermore, 0.5 is the “theoretical value” a model would have if it classed instances randomly. To summarize, the more the area under the ROC curve exceeds 0.5 (up to 1), the better is the model.

Pharmacophore Models.

These models have been used to obtain a three dimensional view of the important groups of atoms, which are linked to the repellent activity. The principle of this method is to use a series of molecules with a known activity in order to point out which groups (or steric, electronic or physico chemical features) are common to all active molecules (i.e., with the highest repellency index) and essential for their biological activities. Usually, six different types of properties are identified: 1) hydrogen bond donor; 2) hydrogen bond acceptor; 3) positive charge; 4) negative charge; 5) aromatic; and 6) hydrophobic. The Catalyst software (Accelrys) was used to generate the instant pharmacophore models. In addition to the six types of groups described above, Catalyst can also look for different properties in the given set of molecules. For instance, groups showing a hydrophobic property can be classified as hydrophobic aromatic or aliphatic. For each model, the maximum number of different chemical features looked at by the software is five. The user can define how many features of each kind, and in total, can be used in a model generation. It is also possible to fix a minimum number for each feature in order to force the recognition of one kind.

The purpose of this study was to extract the supramolecular information in order to establish a map of the “active” chemical functions, and thereby, a relationship between structure and activity. The models have been generated using two different sets of molecules: set 1) all the 50 molecules from the dataset with a positive repellency index (RI); and set 2) only the molecules with a coumarin scaffold and a RI>0.5 (34 molecules). In each case, the data were divided among two subsets: one training set and one test set.

The Catalyst software considers that the compounds with the lowest activity values are the most active (because it is calibrated to be used with a biological activity). Moreover, the software is not able to deal with a logarithmic scale, so it was necessary to introduce a different scale of values for the repellency index that would have a “numerical similarity” with a biological activity. So the scale transfer has been done using Eq.5.

RI=10^{-10 RI} (Eq.5)

To generate a pharmacophore model, the software has to superimpose the different structures in order to see if a feature is present in a sufficient amount of active molecules to be considered as chemically relevant. As it has been described previously, the structures used were all obtained with an energy minimization. But the exclusive use of these structures would not have given any exploitable results, because it is statistically unlikely that the superimposition of these conformations would have led to the generation of a pharmacophore model. Therefore, for each molecule a conformational study has been performed at the molecular mechanics level of calculation using the CHARMM force field (Chemistry at HARvard Macromolecular Mechanics; Brooks Journal of Computable chemistry 1983). The presence of different conformations allows the software to take different “poses” into account when generating the pharmacophore model. To generate the conformation models, the following parameters were used: 1) maximum 250 conformers per compound; 2) use of “Best Quality” criterion; and 3) energy range of 20 kcal/mol.

In general, Catalyst first calculates the costs of two theoretical hypotheses, namely, the ideal hypothesis (fixed cost) and the null hypothesis. The ideal hypothesis has a minimal error cost, and the slope of the activity correlation is one. The null hypothesis has a maximal error cost, and the slope of the activity correlation is zero. Together they represent the upper and lower bounds on cost for the hypotheses that are generated. The greater the difference between them, the greater is the likelihood that a meaningful hypothesis can be found. The closer the cost of the generated hypothesis is to that of the ideal hypothesis, the higher the probability that the generated hypothesis represents a true correlation in the data. The hypothesis with the least cost ideally would map to all the features of the most active compounds in the training set. The cost is reported in bits and a difference of about 50-60 bits between the generated hypotheses and the null hypothesis suggests that the correlation may be significant which in turn requires a difference of about 60-70 bits between the costs of the ideal and null hypotheses (Kristam, 2005).

Results.

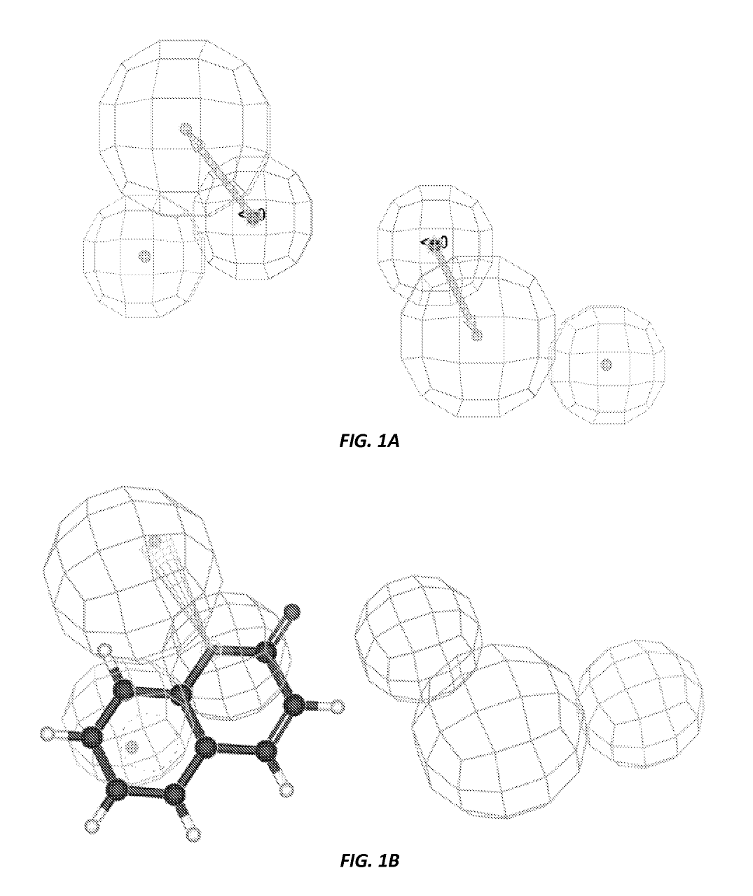

Approximately 25 models were generated each using different input parameters to obtain the model that shows the best correlation between the chosen training and test sets. One useful model was obtained using the “reduced” dataset, containing only coumarins with a repellency index (RI) of 0.5 and more. The training set contained 24 molecules, and the test set contained the remaining 10 molecules. The model has been obtained with the following input parameters: 1) minimum spacing between the features=50 pm; 2) minimum number of features=1; and 3) maximum number of feature=10. The 3D pharmacophore generated (FIG. 1) presents four features, two being hydrogen bonds donors and two being hydrophobic.

The analysis of FIG. 1 shows a possible preferred position for the side chain in 4. This statement is in agreement with the analysis of the data chart. Indeed, considering only the molecules with a coumarin scaffold, it is important to note that the six most repellent compounds (RI≧0.93) possess their side chain in positions 4 and 6 of the bicycle (4 on position 4 and 2 on position 6).

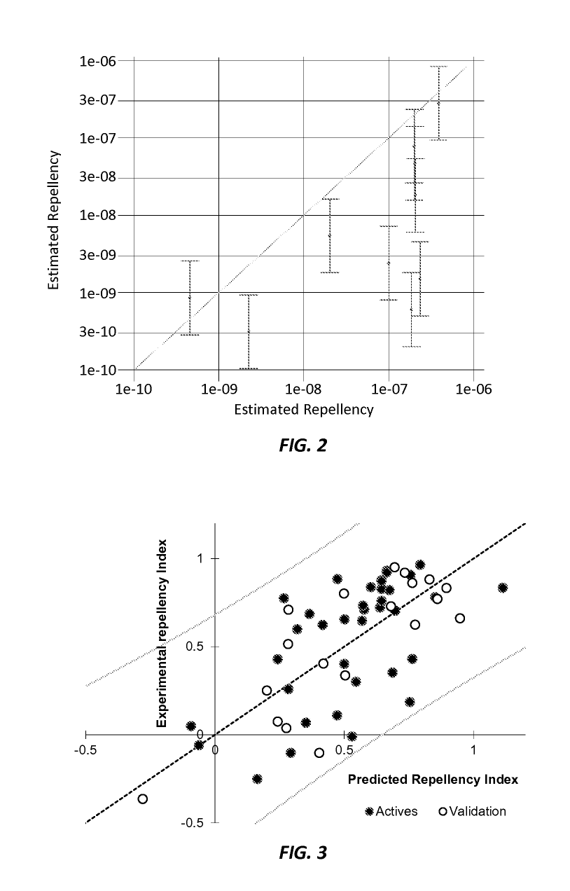

The previously described model has been tested on a subset of 10 coumarins (also with a RI above 0.5); the results are displayed on FIG. 2. It appears that the model has the tendency to slightly underestimate the repellent activity of the molecules. The statistical parameters for this model are: an R² of 0.72, a Q² of 0.45, and a score difference between the null hypothesis and the chosen hypothesis of 32.28.

Linear and Classification Models.

XL-STAT (Addinsoft, 2011) and Weka (IH Witten et al., 2011) were used to generate the decision trees and other linear regressions. The first models were generated using 57 molecules (38 in the training set, 19 in the test). The results of three models obtained are presented in Table 4. The best Q² for a “3 descriptors” model has a value of 0.28. The average R² for all the 3 descriptors models has a value of 0.54 and the average Q² is 0.22. The results indicated that the chosen descriptors produced poor model strength.

TABLE 4
Best model parameters for the 57 coumarins. Linear
regression obtained with three different test sets

| Descriptors | R² | Q² | R²_{adj} | s | F | N |
|---|---|---|---|---|---|---|
| Molecular Solubility // | 0.66 | 0.21 | 0.61 | 0.05 | 12.40 | 38 |
| CHI_0 // CHI_2 // | | | | | | |
| MFPSASA // Shadow XY | | | | | | |
| CHI_0 // CHI_1 // CHI_2 // | 0.41 | 0.65 | 0.34 | 0.10 | 5.71 | 38 |
| MFPSASA | | | | | | |
| CHI_0 // CH1_2 // MFPSASA | 0.62 | 0.12 | 0.59 | 0.06 | 18.47 | 38 |

According to FIG. 3, the “four descriptors” model appears to be both overestimating and underestimating the experimental repellency index (RI) values. In an effort to improve the results, two normalizations were performed; one in the interval [−1; 1] and the other one in the interval [0; 1]. This did not markedly improve the results. Next, new regression models were built using only the 46 molecules having RI>0.1. No stronger models resulted.

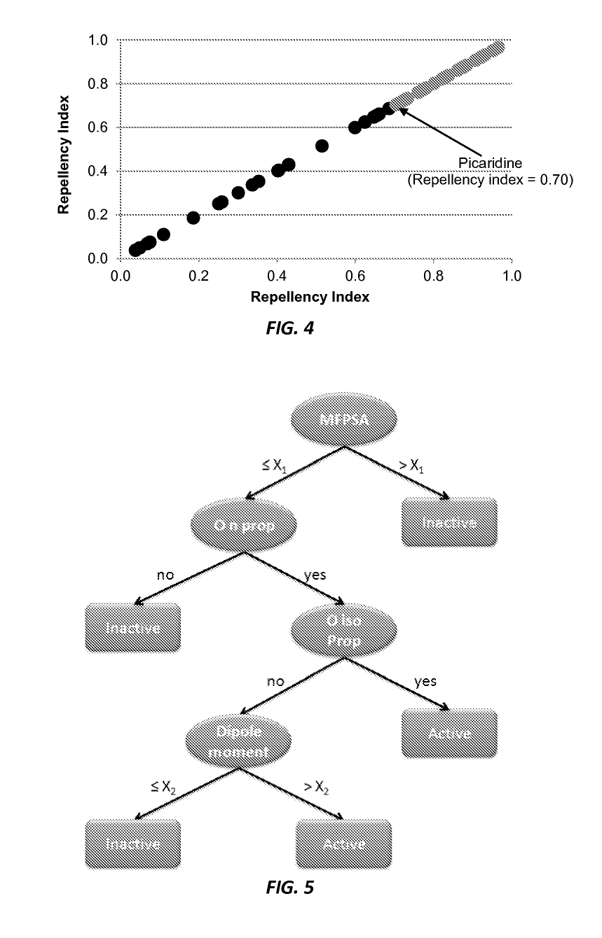

To better understand how particular attributes of the molecule set might be influencing the models, a projection of the compounds depending on their repellency index was performed (FIG. 4). The graph indicates that there are as many molecules in the part where the repulsion index is superior at 0.7 as there are in the rest of the graph. Also, several blank zones can be seen. This pattern of heterogeneous distribution can have the effect of “overtraining” the model in the “strong repellency zone”. Further compounding the problem, it was determined that the repellency index measurement error was about ±0.1. To minimize the deleterious effects of these potentially confounding factors, “repellency classes” were established. Based on the repellency index values, the dataset was divided into 2, 3, 4 or 5 repellency class subsets, in order to reduce the difficulty of modeling the property of slightly repellent molecules. For example, in the four classes' models, the thresholds between the classes are defined as follow:

0.10≦RI<0.50→“Low” repellency class

0.50≦RI<0.70→“Medium” repellency class

0.70≦RI<0.83→“Picaridine” repellency class (0.70 is the RI for Picaridine)

0.83≦RI<1.00→“DEET” repellency class (0.83 is the repellency index for DEET)

The results obtained with the generation of classification trees based on 2, 3, 4 and 5 classes led to the following statements:
(1) there is no better model than the one based on two repellency classes;
(2) the models with 3, 4 and 5 classes do not show a better prediction rate than random classification models;
(3) some of the descriptors used in the decision trees are also used to built the regression models (MFPSA is the most selected descriptor in all the models);
(4) The two classes' model is the most encouraging one and will be taken as starting point for the following studies. It can be used as a filter to screen the repellency of new molecules. Indeed, it could separate the “active” compounds from the rest of the dataset.

Selection Model.

A complete set of experiments, involving different combinations of options (approximately 50 models have been generated), led to the exploitation of one model. The statistical parameters are described in Table 5 and the tree is presented in FIG. 5. It has been generated using the complete set of coumarin (trained on 32 and tested on 14 molecules). It is important to note the presence of two novel descriptors: “O iso prop” and “O n prop”. Both are fragmental descriptors coding for the presence of an isopropyloxy group and an n-propyloxy groups on the side chain, respectively.

TABLE 5
Statistical parameters for the most effective classification tree

| | Correctly classified | Area under |
|---|---|---|
| | instances | ROC curve |
| Training | 91% | 0.94 |
| Internal Leave One Out | 75% | 0.77 |
| External Validation | 71% | 0.78 |

The presented classification pattern will be used to separate the molecules in two classes. The threshold between the “active” and “inactive” molecules has been set to a repellency index value of 0.75, which is a relatively high value. It was chosen in order to balance the number of molecules among the two classes (23 coumarins in each).

Refinement Models.

Three different linear models obtained with the software XLSTAT appeared to be usable, though none showed a very good correlation coefficient for regression (R²) or prediction (Q²). However, the error between the experimental and predicted values was very low, indicating the models possessed strong predictive power.

TABLE 6
Statistical parameters for the linear model generated on the
reduced set of 34 coumarins (RI > 0.5) n = 23.

| | Coefficients (R², R²_{LOO,} Q²) | RMSE |
|---|---|---|
| Training | 0.65 | 0.07 |
| Internal Leave One Out | 0.50 | 0.09 |
| External Validation | 0.71 | 0.05 |

The first model has been obtained by working with a reduced dataset that contains the 34 coumarins with a repellency index of 0.5 and more. It has been trained on 23 molecules and tested on 11. Three descriptors were taken into account: the molecular fractional polar surface area, the presence of an isopropyloxy group and the position of the side chain in C7, which (the last two) are novel descriptors, designed only for this study. The statistical parameters are listed in Table 6 and the results are presented on FIG. 6.

Considering only the RMSE, this model could be used to predict the activity from a set of new molecules. Moreover the analysis of FIG. 6 shows a capacity for the model to predict with more precision the molecules with a high repellency index. The last remark leads to the creation of an even smaller dataset, containing only coumarins with a repellency index of 0.7.

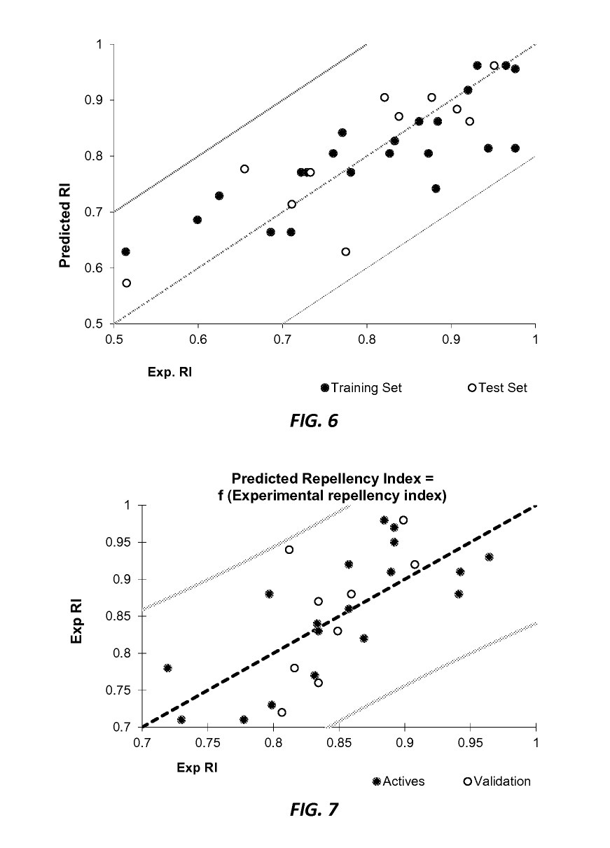

With this dataset, two models give results that sound promising. The first one (FIG. 7) takes three descriptors into account: the position of the side chain in C7, the logarithm of the octanol water partition coefficient (log P or log K_{ow}) and the number of rotatable bonds. The second one (FIG. 8) uses four descriptors: the position of the side chain in C6, the position of the side chain in C7, the “molecular fractional polar surface area” and the presence of an isopropyloxy group. Without going further it is important to note that descriptors like C7 and isopropyloxy are often found in a large number of generated models. The statistical parameters from these two models are given in Table 7. From the analysis of this table, the weakness of the different correlation coefficient is noteworthy; however the RMSE values are low enough so that the results can be considered as exploitable.

TABLE 7
Statistical parameters for the linear models generated on the
reduced set of 28 coumarins (RI > 0.7), n_{training} = 19, nₜₑₛₜ = 9.

| | 3 descriptor model | | 4 descriptor model | |
|---|---|---|---|---|
| | Coefficients | | Coefficients | |
| | (R², R²_{LOO,} Q²) | RMSE | (R², R²_{LOO,} Q²) | RMSE |
| Training | 0.53 | 0.06 | 0.58 | 0.06 |
| Internal Leave | 0.26 | 0.07 | 0.14 | 0.09 |
| One Out | | | | |
| External | 0.69 | 0.06 | 0.42 | 0.08 |
| Validation | | | | |

In parallel to the generation of linear regression based models, some classification trees were built to refine the results obtained with the model described above. With this procedure it was possible to introduce a graduation in the repellency. The purpose is to be able to differentiate the very active compounds from the less active. The statistical parameters from the three models presented are listed in the Table 8.

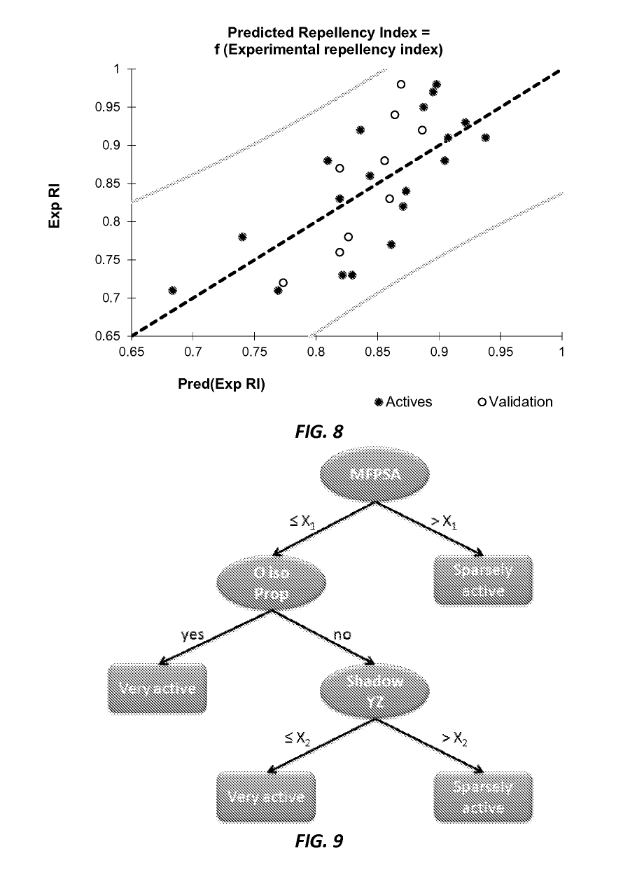

The first model presented has been generated on the dataset containing only the coumarins with repellency index higher than 0.5. It divides the data in two classes: very active (RI>0.80) and sparsely active (0.5<RI<0.80). The obtained tree is presented in the FIG. 9. There are 3 descriptors used, among which can be found the “Molecular Fractional Polar Surface Area” and the descriptor coding for the presence of an isopropyloxy group. Both are found in a large amount of generated models. The last descriptor used is a “Shadow index”, which is relative to the size and shape of the molecules (it codes for the area covered by the “shadow” created by a molecule in a plan) it this case the plan YZ).

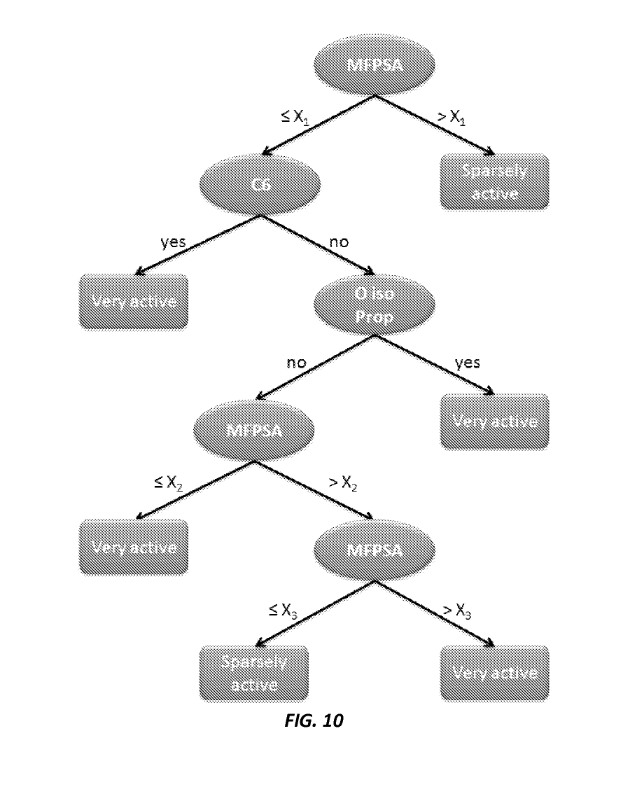

The model seems to be very efficient on a statistical point of view, and therefore, could be trusted to be applied on a larger scale. The second model (FIG. 10) presented has been obtained working on the same set of 34 coumarins (repellency index >0.5). It is the best from the three regression trees designed; indeed its statistical parameters are very good for every aspect tested. It also divides the data in the two same classes as before. The MFPSA is present once again but this time, it appears more than once. This means that the polarity of a compound has to be neither too high nor too low, in order to provide a good repellency. Another “homemade” descriptor appears in the model: C6 with codes the information: “is the side chain on position 6?” A rapid overview from the compounds in the dataset revealed that among the 11 coumarins having a side chain on position 4 or 6, 8 are among the 12 most repellent compounds.

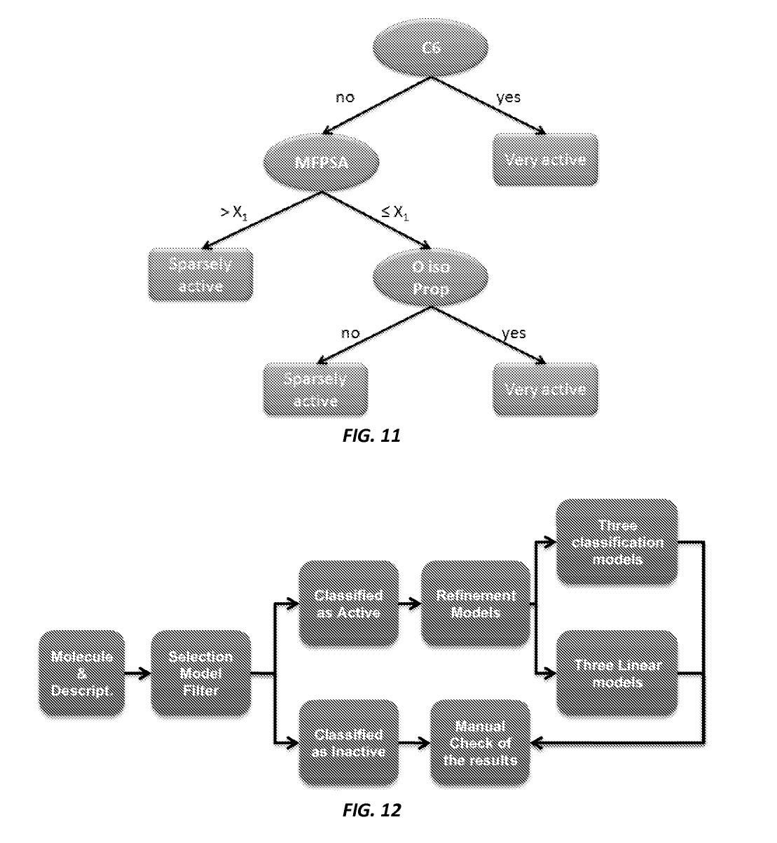

The third model (FIG. 11) has been generated using 28 coumarins with a repellency index higher than 0.7, but the results seem to Indicate that despite the small number of descriptors used, the model is over fitted. Different pruning techniques have been tried in order to change the form of the tree, but is appeared that it was the best results that could be obtained with this set of molecules. The tree divides the data in two classes: very active (RI>0.85) and sparsely active (0.7<RI<0.85).

TABLE 8
Statistical parameters for the classification trees generated on the reduced set of 28
coumarins (RI > 0.7, n = 19), and the reduced set of 34 coumarins (RI > 0.5, n = 23)

| | N = 23, three descriptors (FIG. 9) | | N = 23, five descriptors (FIG. 10) | | N = 19, three descriptors (FIG. 11) | |
|---|---|---|---|---|---|---|
| | Correctly classified | Area under | Correctly classified | Area under | Correctly classified | Area under |
| | instances | ROC curve | instances | ROC curve | instances | ROC curve |
| Training | 87% | 0.90 | 91% | 0.95 | 89% | 0.92 |
| Internal | 57% | 0.54 | 65% | 0.55 | 36% | 0.50 |
| Leave One | | | | | | |
| Out | | | | | | |
| External | 73% | 0.90 | 82% | 0.90 | 66% | 0.58 |
| Validation | | | | | | |

To summarize what has been done regarding the generation of QSAR models, it is important to note that two different aspects of the problem have been treated. With the help of the first presented regression tree, it is possible to predict if the repellent activity of a new compound will be higher than 0.75 (active) or not (inactive). Depending upon this result, the molecules will be tested with the other models in order to obtain a more precise estimate of its repellency, either in terms of a new class (very active or sparsely active), or with a numeric value. The obtained trees also allow having a general point of view of how the repellency of a molecule is affected with some changes performed among its structure. Using this approach, several sets of new molecules have been created and their repellency has been predicted.

### Example 2

### Novel Repellent Molecules Revealed by Repellency Models

Assisted by the descriptors and the above-disclosed models, a class has been attributed to each new compound. The way the new molecules' activities were predicted is presented on FIG. 12. Firstly, the selection model from FIG. 5 was applied; it allows deciding if the compound is active or inactive. If it was classified as active, the six “refinement” models were applied (models from FIGS. 6 to 11). The results obtained with these models give information on “how active is a compound?”

Using the models presented in Example 1, repellent activity was predicted for novel molecules (Table 9). Four different sets of molecules have been generated. The first one is composed of 10 molecules (from PN10001 to PN10010) created by observing the most active compounds and changing the position of the side chain; for example if the side chain is in position 7 on the compound with the known activity, the molecules generated have their side chain in the positions 4 and 6. The second is composed with molecules issue from former studies done by Nathalie GENESTE (from NG1 to NG8). The third has been composed after the results obtained with the molecules from the second set (from PN10011 to PN10022). The last set (from PN10031 to PN10056) was generated after discussion with all the group members.

For all molecules, the preparative work is the same as the one described in the previous report. This means, a non optimized tridimensional structure was generated, followed with a conformational analysis (performed with the commercial software AMPAC). Then the lowest energy conformation has been extracted in order to perform an energy minimization with the HF/6-31G* method; the software GAUSSIAN was used for this purpose. Once the optimized structure was obtained, the molecular descriptors were calculated.

For each molecule created, the selection model has been applied, if the compound is classified as active, the 6 other models are applied; if not nothing more should be done with the molecule. But the six models were still applied in order to be able to check if all the results are in agreement with each other. It appears that it is not true for all compounds; indeed some predictions indicate that one molecule classified as inactive by the selection model could still have good repelling properties. This is the case for NG4, which has been classified as inactive and three times as sparsely active, but the three numeric values predicted indicate the compound could exhibit a high repellency value. It especially presents a substituent on position 8; following this idea the third set of molecules was generated. The predicted results from the third set are very similar to those from the second one. Indeed, all the molecules were predicted to be inactive but three molecules out of the twelve present results within the refined models that are in contradiction with the selection model: PN 10014, PN 10015, and PN 10016.

The structures of the three molecules are similar and present a dibutyl amide on position 6 (of the coumarin cycle). The same group was found on molecule NG4 but on position 7 (of the coumarin cycle). The three molecules differ from each other by the substituent present on position 8 (H for PN 10014, CH₃ for PN 10015, and Cl for PN 10016). Such a substituent was also present on NG4 (Cl on position 8).

TABLE 9
predicted repellency of novel compounds

| | | Model-Predicted Repellency Value | | | | | | |
|---|---|---|---|---|---|---|---|---|
| | | | 1st | 2nd | 3rd | 1st | 2nd | 3rd |
| ID | Structure | Select | Lin. | Lin. | Lin. | tree | tree | tree |
| PN10001 | 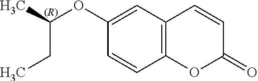 | Active | 0.92 | 0.91 | 0.94 | Very Active | Very Active | Very Active |
| PN10002 | 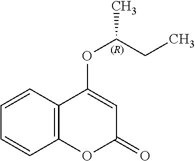 | Active | 0.92 | 0.88 | 0.87 | Very Active | Very Active | Sparsely Active |
| PN10003 | 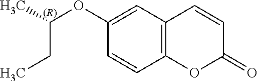 | Active | 0.92 | 0.91 | 0.94 | Very Active | Very Active | Very Active |
| PN10004 | 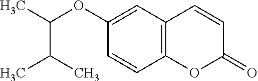 [SMILES: CC(C)C(C)Oc1ccc2oc(=O)ccc2c1] | Active | 0.95 | 0.95 | 0.97 | Very Active | Very Active | Very Active |
| PN10005 | 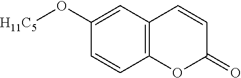 | Active | 0.86 | 0.91 | 0.90 | Very Active | Very Active | Very Active |
| PN10006 | 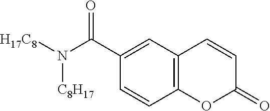 | Inactive | 0.99 | 1.00 | 1.04 | Sparsely active | Very Active | Very Active |
| PN10007 | 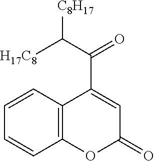 | Inactive | 0.99 | 1.00 | 1.04 | Sparsely active | Sparsely active | Sparsely active |
| PN10008 | 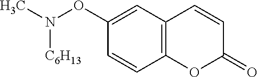 | Active | 1.02 | 0.99 | 1.01 | Very Active | Very Active | Very Active |
| PN10009 | 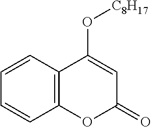 | Active | 0.93 | 0.94 | 0.88 | Sparsely Active | Very Active | Sparsely Active |
| PN10010 | 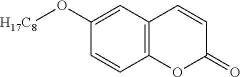 | Active | 0.93 | 0.97 | 0.96 | Sparsely Active | Very Active | Very Active |
| NG1 | 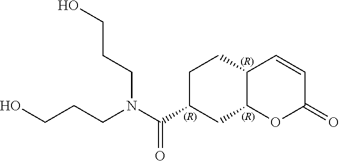 | Inactive | 0.41 | 0.39 | 0.34 | Sparsely active | Sparsely active | Sparsely active |
| NG2 | 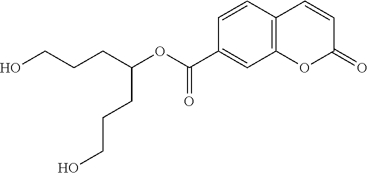 [SMILES: O=C(OC(CCCO)CCCO)c1ccc2ccc(=O)oc2c1] | Inactive | 0.44 | 0.40 | 0.49 | Sparsely active | Sparsely active | Sparsely active |
| NG3 | 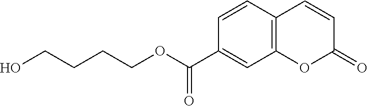 [SMILES: O=C(OCCCCO)c1ccc2ccc(=O)oc2c1] | Inactive | 0.37 | 0.35 | 0.57 | Sparsely active | Sparsely active | Sparsely active |
| NG4 | 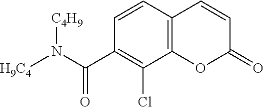 | Inactive | 0.80 | 0.81 | 0.85 | Sparsely active | Sparsely active | Sparsely active |
| NG5 | 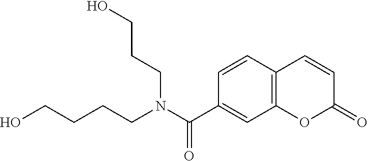 [SMILES: O=C(c1ccc2ccc(=O)oc2c1)N(CCCO)CCCCO] | Inactive | 0.38 | 0.36 | 0.37 | Sparsely active | Sparsely active | Sparsely active |
| NG6 | 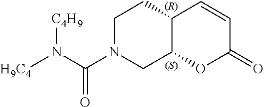 | Inactive | 0.78 | 0.80 | 0.74 | Sparsely active | Sparsely active | Sparsely active |
| NG7 | 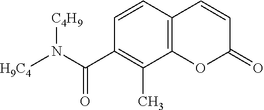 | Inactive | 0.79 | 0.81 | 0.83 | Sparsely active | Sparsely active | Sparsely active |
| NG8 | 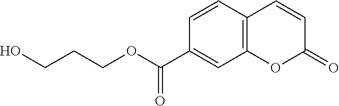 [SMILES: O=C(OCCCO)c1ccc2ccc(=O)oc2c1] | Inactive | 0.32 | 0.29 | 0.53 | Sparsely active | Very Active | Sparsely active |
| PN 10011 | 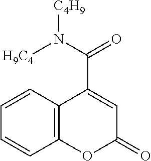 | Inactive | 0.87 | 0.87 | 0.88 | Sparsely active | Sparsely active | Sparsely active |
| PN10012 | 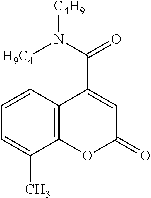 | Inactive | 0.89 | 0.90 | 0.93 | Sparsely active | Sparsely active | Sparsely active |
| PN10013 | 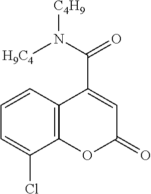 | Inactive | 0.90 | 0.90 | 0.95 | Sparsely active | Sparsely active | Sparsely active |
| PN10014 | 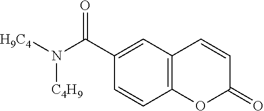 | Inactive | 0.87 | 0.87 | 0.88 | Sparsely active | Very Active | Very Active |
| PN10015 | 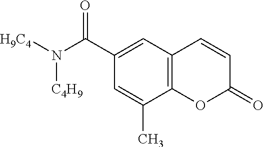 | Inactive | 0.89 | 0.90 | 0.94 | Sparsely active | Very Active | Very Active |
| PN10016 | 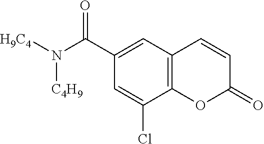 | Inactive | 0.90 | 0.90 | 0.95 | Very Active | Very Active | Very Active |
| PN10017 | 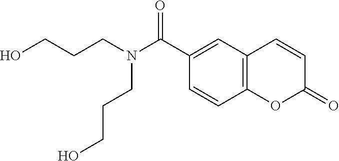 [SMILES: O=C(c1ccc2oc(=O)ccc2c1)N(CCCO)CCCO] | Inactive | 0.48 | 0.44 | 0.48 | Sparsely active | Sparsely active | Very Active |
| PN10018 | 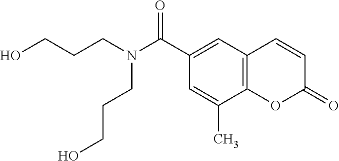 [SMILES: Cc1cc(C(=O)N(CCCO)CCCO)cc2ccc(=O)oc12] | Inactive | 0.53 | 0.50 | 0.53 | Sparsely active | Sparsely active | Very Active |
| PN10032 | 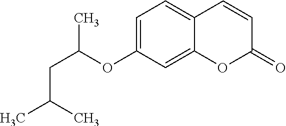 [SMILES: CC(C)CC(C)Oc1ccc2ccc(=O)oc2c1] | Active | 0.88 | 0.85 | 0.88 | Very Active | Very Active | Very Active |
| PN10033 | 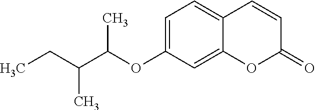 [SMILES: CCC(C)C(C)Oc1ccc2ccc(=O)oc2c1] | Active | 0.88 | 0.86 | 0.88 | Very Active | Very Active | Very Active |
| PN10034 | 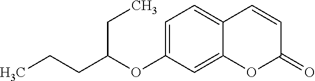 [SMILES: CCCC(CC)Oc1ccc2ccc(=O)oc2c1] | Active | 0.88 | 0.85 | 0.88 | Very Active | Very Active | Very Active |
| PN10035 | 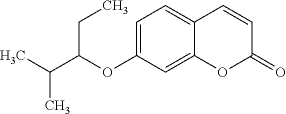 [SMILES: CCC(Oc1ccc2ccc(=O)oc2c1)C(C)C] | Active | 0.88 | 0.87 | 0.88 | Very Active | Very Active | Very Active |
| PN10036 | 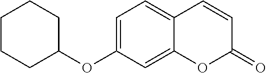 [SMILES: O=c1ccc2ccc(OC3CCCCC3)cc2o1] | Active | 0.84 | 0.90 | 0.85 | Very Active | Very Active | Sparsely active |
| PN10037 | 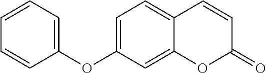 [SMILES: O=c1ccc2ccc(Oc3ccccc3)cc2o1] | Active | 0.82 | 0.87 | 0.83 | Very Active | Very Active | Sparsely active |
| PN10038 | 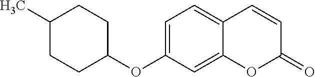 [SMILES: CC1CCC(Oc2ccc3ccc(=O)oc3c2)CC1] | Active | 0.87 | 0.93 | 0.87 | Very Active | Very Active | Very Active |
| PN10039 | 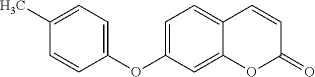 [SMILES: Cc1ccc(Oc2ccc3ccc(=O)oc3c2)cc1] | Active | 0.86 | 0.92 | 0.86 | Very Active | Very Active | Very Active |
| PN10040 | 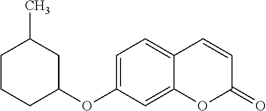 [SMILES: CC1CCCC(Oc2ccc3ccc(=O)oc3c2)C1] | Active | 0.87 | 0.93 | 0.87 | Very Active | Very Active | Very Active |
| PN10041 | 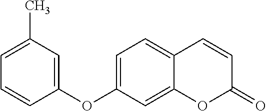 [SMILES: Cc1cccc(Oc2ccc3ccc(=O)oc3c2)c1] | Active | 0.86 | 0.92 | 0.86 | Very Active | Very Active | Very Active |
| PN10042 | 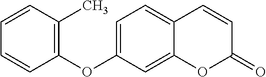 [SMILES: Cc1ccccc1Oc1ccc2ccc(=O)oc2c1] | Active | 0.85 | 0.92 | 0.86 | Very Active | Very Active | Very Active |
| PN10043 | 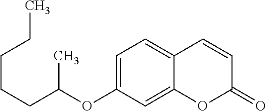 [SMILES: CCCCCC(C)Oc1ccc2ccc(=O)oc2c1] | Active | 0.90 | 0.86 | 0.89 | Very Active | Very Active | Very Active |
| PN10044 | 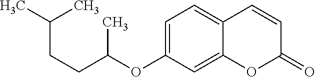 [SMILES: CC(C)CCC(C)Oc1ccc2ccc(=O)oc2c1] | Active | 0.90 | 0.87 | 0.89 | Very Active | Very Active | Very Active |
| PN10045 | 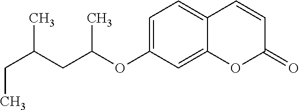 [SMILES: CCC(C)CC(C)Oc1ccc2ccc(=O)oc2c1] | Active | 0.90 | 0.87 | 0.89 | Very Active | Very Active | Very Active |
| PN10046 | 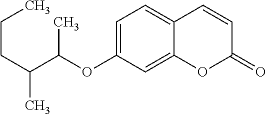 [SMILES: CCCC(C)C(C)Oc1ccc2ccc(=O)oc2c1] | Active | 0.90 | 0.88 | 0.89 | Very Active | Very Active | Very Active |
| PN10047 | 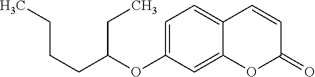 [SMILES: CCCCC(CC)Oc1ccc2ccc(=O)oc2c1] | Active | 0.90 | 0.87 | 0.89 | Very Active | Very Active | Very Active |
| PN10048 | 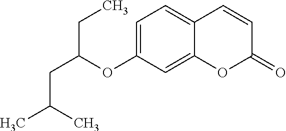 [SMILES: CCC(CC(C)C)Oc1ccc2ccc(=O)oc2c1] | Active | 0.90 | 0.88 | 0.89 | Very Active | Very Active | Very Active |
| PN10049 | 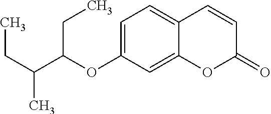 [SMILES: CCC(C)C(CC)Oc1ccc2ccc(=O)oc2c1] | Active | 0.90 | 0.89 | 0.89 | Very Active | Very Active | Very Active |
| PN10050 | 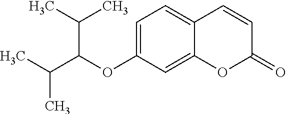 [SMILES: CC(C)C(Oc1ccc2ccc(=O)oc2c1)C(C)C] | Active | 0.91 | 0.90 | 0.90 | Very Active | Very Active | Very Active |
| PN10051 | 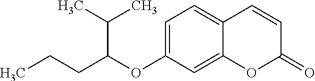 [SMILES: CCCC(Oc1ccc2ccc(=O)oc2c1)C(C)C] | Active | 0.90 | 0.89 | 0.89 | Very Active | Very Active | Very Active |
| PN10052 | 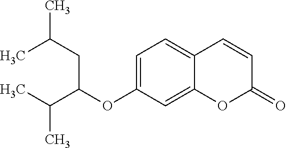 [SMILES: CC(C)CC(Oc1ccc2ccc(=O)oc2c1)C(C)C] | Active | 0.93 | 0.91 | 0.91 | Very Active | Very Active | Very Active |
| PN10053 | 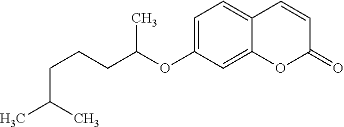 [SMILES: CC(C)CCCC(C)Oc1ccc2ccc(=O)oc2c1] | Active | 0.93 | 0.89 | 0.91 | Very Active | Very Active | Very Active |
| PN10054 | 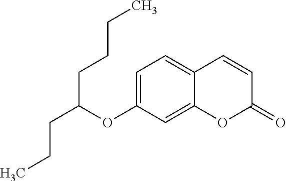 [SMILES: CCCCC(CCC)Oc1ccc2ccc(=O)oc2c1] | Active | 0.92 | 0.89 | 0.91 | Very Active | Very Active | Very Active |
| PN10055 | 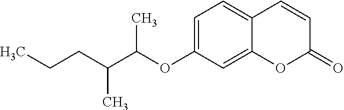 [SMILES: CCCC(C)C(C)Oc1ccc2ccc(=O)oc2c1] | Active | 0.90 | 0.88 | 0.89 | Very Active | Very Active | Very Active |
| PN10056 | 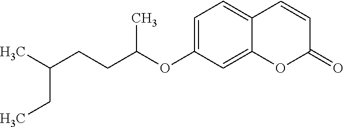 [SMILES: CCC(C)CCC(C)Oc1ccc2ccc(=O)oc2c1] | Active | 0.93 | 0.89 | 0.91 | Very Active | Very Active | Very Active |

This contradiction between the models can be explained by the chemical nature of these molecules. For example, the molecular fractional polar surface area for some compounds in Table 8 is much higher than the values found in the original dataset. Further, no compound used to build the model had a side chain on position 8. In the first set of compounds, eight molecules were classified as active, among this subset, one compound is particularly interesting (predicted “active” by all models): PN110008. It has a side chain in position 6, with an isopropyloxy group and a seven carbon long hydrophobic chain. However, it has been demonstrated molecules modified at position 7 tend to be effective longer than do those modified at either position 6 or 4.

In view of above, a new set of 25 molecules has been designed (PN10031-PN10056), which contains exclusively coumarin derivatives substituted on position 7 with an isopropyloxy group. All the molecules are predicted to be active and a large majority (23/25) is predicted 3 times to be very active. The predicted RI values range from 0.83 to 0.94 which is quite high and so these molecules will advance to in vivo studies.

Also of interest are compounds that were predicted to be inactive by the selection model, but active via the other models. For example, PN 10014, PN 10015, and PN 10016 present the same scaffold, with only the substituent in position 8 varying (respectively: H, CH₃, and Cl). The common dibutylamide group suggests this particular scaffold might be a promising repellent core upon which build.

### PROPHETIC EXAMPLE

Compounds of the instant disclosure are applied to animals and surroundings to repel pests.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A compound according to formula (I) or (Ia): 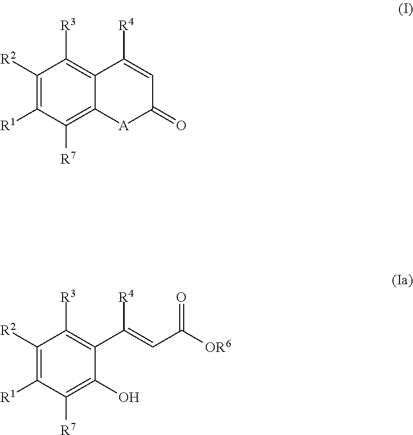
wherein
R¹, R², R³, R⁴, and R⁶ independently include H, C, OR⁵, CR⁵, OCC(═O)N(R⁵)(R⁸), CC(═O)N(R⁵)(R⁸), alkyl, aryl, aralkyl, heteroaryl, alcohols, amine, aldehyde, heterocyclyl, or salts of amines and carboxylates;
A is O, or the ring is opened at A, thus forming compounds according to formula (Ia);
R⁵ and R⁸ are independently selected from alkyl, aryl, aralkyl, heteroaryl, alcohols, amine, aldehyde, or heterocyclyl; or R⁵ and R⁸ come together to form a C₃-C₁₀ ring, which may be aromatic, non-aromatic, and/or substituted with alkyl, aryl, aralkyl, heteroaryl, alcohols, amine, aldehyde, heterocyclyl, or combinations thereof.

2. The compound of claim 1 which is PN10001, PN10002, PN10003, PN10004, PN10005, PN10006, PN10007, PN10008, PN10009, PN10010, NG1, NG2, NG3, NG4, NG5, NG6, NG7, NG8, PN10011, PN10012, PN10013, PN10014, PN10015, PN10016, PN10017, PN10018, PN10019, PN10020, PN10021, PN10022, PN10031, PN10032, PN10033, PN10034, PN10035, PN10036, PN10037, PN10038, PN10039, PN10040, PN10041, PN10042, PN10043, PN10044, PN10045, PN10046, PN10047, PN10048, PN10049, PN10050, PN10051, PN10052, PN10053, PN10054, PN10055, or PN10056.

3. The compound of claim 1 wherein:
A=O;
R²═R³═R⁴ is H;
R¹ is OC(═O)OR⁵;
R⁵ is alkyl, alkenyl, or alkynyl; and
R⁷ is H, alkyl, alkenyl, or halogen.

4. The compound of claim 1 wherein
A=O;
R²═R³═R⁴ is H;
R¹ is OC(═O)OR⁵;
R⁵ is alkyl; and
R⁷ is H, alkyl, alkenyl, or halogen.

5. The compound of claim 1 wherein
A=O;
R²═R³═R⁴ is H;
R¹ is OC(═O)R⁵;
R⁵ is alkyl, alkyl ether, CCOC(═O)C, alkyl acetate, alkenyl, alkenyl acetate, alkynyl or alkynyl acetate; and
R⁷ is H, alkyl, alkenyl, or halogen.

6. The compound of claim 1 wherein:
A=O
R¹═R²═R³ is H;
R⁴ is OC(═O)OR⁵;
R⁵ is alkyl, alkenyl, or alkynyl; and
R⁷ is H, alkyl, alkenyl, or halogen.

7. The compound of claim 1 wherein
A=O;
R¹═R²═R³ is H;
R⁴ is OC(O)OR⁵;
R⁵ is alkyl; and
R⁷ is H, alkyl, alkenyl, or halogen.

8. The compound of claim 1 wherein
A=O
R¹═R²═R³ is H;
R⁴ is OC(═O)R⁵;
R⁵ is alkyl, alkyl ether, CCOC(═O)C, alkyl acetate, alkenyl, alkenyl acetate, alkynyl, or alkynyl acetate; and
R⁷ is H, alkyl, alkenyl, or halogen.

9. The compound of claim 1 wherein:
A=O;
R¹═R²═R³ is H;
R⁴ is C(═O)OR⁵;
R⁵ is alkyl, alkenyl, or alkynyl; and
R⁷ is H, alkyl, alkenyl, or halogen.

10. The compound of claim 1 wherein:
A=O;
R¹═R²═R³ is H;
R⁴ is OR⁵;
R⁵ is alkyl, alkenyl, or alkynyl; and
R⁷ is H, alkyl, alkenyl, or halogen.

11. The compound of claim 1 wherein:
A=O;
R⁷═R³═R⁴ is H;
R¹ is OCC(═O)N(R⁵)(R⁸) or CC(═O)N(R⁵)(R⁸);
R⁵ and R⁸ are independently selected from alkyl, alkenyl, or alkynyl; and
R⁷ is H, alkyl, alkenyl, or halogen.

12. The compound of claim 1 wherein:
A=O;
R²═R³═R⁴ is H;
R¹ is OCC(═O)N(R⁵)(R⁸) or CC(═O)N(R⁵)(R⁸);
R⁵ and R⁸ come together to form a C3-C10 ring which may be substituted by alkyl, alkenyl, or alkynyl; and
R⁷ is H, alkyl, alkenyl, or halogen.

13. An insect/pest repellent composition comprising the compound of any one of the proceeding claims.

14. The composition of claim 13 that is in a form suitable for topical application to an animal.

15. The composition of claim 14 that is a cream, gel, spray, liquid or spot-on.

16. A method for repelling pests comprising the step of applying a compound or composition of any of the proceeding claims to animals or a locus.

17. The method of claim 16 wherein the animals are birds or mammals.

18. The method of claim 17 wherein the mammals are humans, equines, felines, canines, bovines, or caprines.

19. The method of 18 wherein the animals are equines or bovines.

20. The method of 18 wherein the animals are humans.
